SEQUENCE LISTING (14 sequences)

SEQ ID NO: 1 - protein, 685 aa, artificial
  luciferase fusion protein LuxAB
MKFGNFLLTYQPPELSQTEVMKRLVNLGKASEGCGFDTVWLLEHHFTEFGLLGNPYVAAAHLLGATETLNVGTAAIVLPTAHPVRQAEDVNLLDQMSKGRFRFGICRGLYDKDFRVFGTDMDNSRALMDCWYDLMKEGFNEGYIAADNEHIKFPKIQLNPSAYTQGGAPVYVVAESASTTEWAAERGLPMILSWIINTHEKKAQLDLYNEVATEHGYDVTKIDHCLSYITSVDHDSNRAKDICRNFLGHWYDSYVNATKIFDDSDQTKGYDFNKGQWRDFVLKGHKDTNRRIDYSYEINPVGTPEECIAIIQQDIDATGIDNICCGFEANGSEEEIIASMKLFQSDVMPYLKEKQYLIFSQKERDKKFGLFFLNFMNSKRSSDQVIEEMLDTAHYVDQLKFDTLAVYENHFSNNGVVGAPLTVAGFLLGMTKNAKVASLNHVITTHHPVRVAEEACLLDQMSEGRFAFGFSDCEKSADMRFFNRPTDSQFQLFSECHKIINDAFTTGYCHPNNDFYSFPKISVNPHAFTEGGPAQFVNATSKEVVEWAAKLGLPLVFRWDDSNAQRKEYAGLYHEVAQAHGVDVSQVRHKLTLLVNQNVDGEAARAEARVYLEEFVRESYSNTDFEQKMGELLSENAIGTYEESTQAARVAIECCGAADLLMSFESMEDKAQQRAVIDVVNANIV

SEQ ID NO: 2 - protein, 691 aa, artificial
  luciferase fusion protein LuxBA_1
MKFGLFFLNFMNSKRSSDQVIEEMLDTAHYVDQLKFDTLAVYENHFSNNGVVGAPLTVAGFLLGMTKNAKVASLNHVITTHHPVRVAEEACLLDQMSEGRFAFGFSDCEKSADMRFFNRPTDSQFQLFSECHKIINDAFTTGYCHPNNDFYSFPKISVNPHAFTEGGPAQFVNATSKEVVEWAAKLGLPLVFRWDDSNAQRKEYAGLYHEVAQAHGVDVSQVRHKLTLLVNQNVDGEAARAEARVYLEEFVRESYSNTDFEQKMGELLSENAIGTYEESTQAARVAIECCGAADLLMSFESMEDKAQQRAVIDVVANIVTRIDEANQRATKMLGSGMKFGNFLLTYQPPELSQTEVMKRLVNLGKASEGCGFDTVWLLEHHFTEFGLLGNPYVAAAHLLGATETLNVGTAAIVLPTAHPVRQAEDVNLLDQMSKGRFRFGICRGLYDKDFRVFGTDMDNSRALMDCWYDLMKEGFNEGYIAADNEHIKFPKIQLNPSAYTQGGAPVYVVAESASTTEWAAERGLPMILSWIINTHEKKAQLDLYNEVATEHGYDVTKIDHCLSYITSVDHDSNRAKDICRNFLGHWYDSYVNATKIFDDSDQTKGYDFNKGQWRDFVLKGHKDTNRRIDYSYEINPVGTPEECIAIIQQDIDATGIDNICCGFEANGSEEEIIASMKLFQSDVMPYLKEKQ

SEQ ID NO: 3 - protein, 741 aa, artificial
  luciferase fusion protein LuxBA_3
MKFGLFFLNFMNSKRSSDQVIEEMLDTAHYVDQLKFDTLAVYENHFSNNGVVGAPLTVAGFLLGMTKNAKVASLNHVITTHHPVRVAEEACLLDQMSEGRFAFGFSDCEKSADMRFFNRPTDSQFQLFSECHKIINDAFTTGYCHPNNDFYSFPKISVNPHAFTEGGPAQFVNATSKEVVEWAAKLGLPLVFRWDDSNAQRKEYAGLYHEVAQAHGVDVSQVRHKLTLLVNQNVDGEAARAEARVYLEEFVRESYSNTDFEQKMGELLSENAIGTYEESTQAARVAIECCGAADLLMSFESMEDKAQQRAVIDVVRRVTDARENEMDENLEQVSGIIGNLRHMALDMGNEIDTQNRQIDRIMEKADSNKTRIDEANQRATKMLGSGKKFGNFLLTYQPPELSQTEVMKRLVNLGKASEGCGFDTVWLLEHHFTEFGLLGNPYVAAAHLLGATETLNVGTAAIVLPTAHPVRQAEDVNLLDQMSKGRFRFGICRGLYDKDFRVFGTDMDNSRALMDCWYDLMKEGFNEGYIAADNEHIKFPKIQLNPSAYTQGGAPVYVVAESASTTEWAAERGLPMILSWIINTHEKKAQLDLYNEVATEHGYDVTKIDHCLSYITSVDHDSNRAKDICRNFLGHWYDSYVNATKIFDDSDQTKGYDFNKGQWRDFVLKGHKDTNRRIDYSYEINPVGTPEECIAIIQQDIDATGIDNICCGFEANGSEEEIIASMKLFQSDVMPYLKEKQ

SEQ ID NO: 4 - protein, 319 aa, Vibrio fischeri
KFGLFFLNFMNSKRSSDQVIEEMLDTAHYVDQLKFDTLAVYENHFSNNGVVGAPLTVAGFLLGMTKNAKVASLNHVITTHHPVRVAEEACLLDQMSEGRFAFGFSDCEKSADMRFFNRPTDSQFQLFSECHKIINDAFTTGYCHPNNDFYSFPKISVNPHAFTEGGPAQFVNATSKEVVEWAAKLGLPLVFRWDDSNAQRKEYAGLYHEVAQAHGVDVSQVRHKLTLLVNQNVDGEAARAEARVYLEEFVRESYSNTDFEQKMGELLSENAIGTYEESTQAARVAIECCGAADLLMSFESMEDKAQQRAVIDVVNANIV

SEQ ID NO: 5 - protein, 355 aa, Vibrio fischeri
MKFGNFLLTYQPPELSQTEVMKRLVNLGKASEGCGFDTVWLLEHHFTEFGLLGNPYVAAAHLLGATETLNVGTAAIVLPTAHPVRQAEDVNLLDQMSKGRFRFGICRGLYDKDFRVFGTDMDNSRALMDCWYDLMKEGFNEGYIAADNEHIKFPKIQLNPSAYTQGGAPVYVVAESASTTEWAAERGLPMILSWIINTHEKKAQLDLYNEVATEHGYDVTKIDHCLSYITSVDHDSNRAKDICRNFLGHWYDSYVNATKIFDDSDQTKGYDFNKGQWRDFVLKGHKDTNRRIDYSYEINPVGTPEECIAIIQQDIDATGIDNICCGFEANGSEEEIIASMKLFQSDVMPYLKEKQ

SEQ ID NO: 6 - protein, 17 aa, artificial
  SNAP25 linker 1
TRIDEANQRATKMLGSG

SEQ ID NO: 7 - protein, 13 aa, artificial
  SNAP25 linker 2
IDEANQRATKMLG

SEQ ID NO: 8 - protein, 72 aa, artificial
  SNAP25 linker 3
RRVTDARENEMDENLEQVSGIIGNLRHMALDMGNEIDTQNRQIDRIMEKADSNKTRIDEANQRATKMLGSGK

SEQ ID NO: 9 - DNA, 5230 bp, Artificial Sequence
  construct MRZ_LuxAB0; DNA vector comprising LuxAB0 sequence
ccatcgaatggccagatgattaattcctaatttttgttgacactctatcattgatagagttattttaccactccctatcagtgatagagaaaagtgaaatgaatagttcgacaaaaatctagaaataattttgtttaactttaagaaggagatatacaaatgaagttcggcaacttcctgctgacctaccagccccctgagctgagccagaccgaagtgatgaagaggctggtgaacctgggcaaggccagcgagggctgtggcttcgacaccgtgtggctgctggaacaccacttcaccgagttcggcctgctgggcaacccttacgtggccgctgcccatctgctgggcgccaccgagacactgaacgtgggcaccgccgccattgtgctgcctacagcccaccctgtgcggcaggccgaggacgtgaacctgctggaccagatgagcaagggcaggttcagattcggcatctgcaggggcctgtacgacaaggacttcagggtgttcggcaccgacatggacaacagcagggctctgatggactgttggtacgacctgatgaaggaaggcttcaacgagggctacattgccgccgacaacgagcacatcaagttccccaagatccagctgaaccccagcgcctacacacagggcggagcccctgtgtacgtggtggccgagagcgcctctacaaccgagtgggccgctgagaggggcctgcccatgatcctgagctggatcatcaacacccacgagaagaaggcccagctggacctgtacaacgaggtggccacagagcacggctacgacgtgaccaagatcgaccactgcctgagctacatcaccagcgtggaccacgacagcaacagggccaaggacatctgcaggaactttctgggccattggtacgacagctacgtgaacgccaccaagatcttcgacgacagcgaccagaccaagggctacgacttcaacaagggccagtggagggacttcgtgctgaagggccacaaggacaccaacaggcggatcgactacagctacgagatcaaccccgtgggcacccccgaggaatgtatcgccatcatccagcaggacatcgacgccaccggcatcgacaacatctgctgcggcttcgaggccaacggcagcgaggaagagatcattgccagcatgaagctgttccagagcgacgtgatgccctacctgaaagagaagcagtacctgatcttcagccagaaagagagggacaagaagttcgggctgttcttcctgaacttcatgaacagcaagaggtccagcgaccaggtgatcgaggaaatgctggacaccgcccactacgtggaccagctgaagttcgacaccctggccgtgtacgagaaccacttcagcaacaacggcgtggtgggagcccctctgacagtggccggcttcctgctgggaatgaccaagaacgccaaggtggccagcctgaaccacgtgatcaccacccaccatcctgtgcgggtggccgaagaggcctgtctgctggatcagatgtccgagggcagattcgccttcggcttcagcgactgcgagaagtccgccgacatgcggttcttcaacaggcccaccgacagccagttccagctgttcagcgagtgccacaagatcatcaacgacgccttcaccaccggctactgccaccccaacaacgacttctacagcttccctaagatctccgtgaacccccacgcctttacagagggcggacccgcccagttcgtgaacgctaccagcaaggaagtggtggagtgggctgccaagctgggcctgcccctggtgttcagatgggacgactccaacgcccagaggaaagagtacgccggcctgtaccatgaagtggctcaggctcacggcgtggacgtgtcccaggtccggcacaagctgaccctgctggtgaaccagaacgtggacggcgaggccgctagagccgaggccagggtgtacctggaagagttcgtgcgggagagctacagcaacaccgacttcgagcagaagatgggcgagctgctgagcgagaacgccatcggcacctacgaggaaagcacccaggccgccagagtggccatcgagtgctgtggagccgccgacctgctgatgagcttcgagagcatggaagataaggcccagcagagggccgtgatcgacgtggtgaacgccaacatcgtgggtctcagcgcttggagccacccgcagttcgaaaaataataagcttgacctgtgaagtgaaaaatggcgcacattgtgcgacattttttttgtctgccgtttaccgctactgcgtcacggatctccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattgatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaggaattaatgatgtctcgtttagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgcagtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaagcagcataacctttttccgtgatggtaacttcactagtttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgacccgaca

SEQ ID NO: 10 - DNA, 5398 bp, artificial sequences
  construct MRZ_LuxBA3; DNA vector comprising LuxBA3 sequence
ccatcgaatggccagatgattaattcctaatttttgttgacactctatcattgatagagttattttaccactccctatcagtgatagagaaaagtgaaatgaatagttcgacaaaaatctagaaataattttgtttaactttaagaaggagatatacaaatgaagttcggcctgttcttcctgaacttcatgaacagcaagaggtccagcgaccaggtgatcgaggaaatgctggacaccgcccactacgtggaccagctgaagttcgacaccctggccgtgtacgagaaccacttcagcaacaacggcgtggtgggagcccctctgacagtggccggcttcctgctgggcatgaccaagaacgccaaggtggccagcctgaaccacgtgatcaccacccaccatcctgtgcgggtggccgaggaagcctgcctgctggaccagatgagcgagggcagattcgccttcggcttcagcgactgcgagaagtccgccgacatgcggttcttcaacaggcccaccgacagccagttccagctgttcagcgagtgccacaagatcatcaacgacgccttcaccaccggctactgccaccccaacaacgacttctacagcttccccaagatcagcgtgaacccccacgccttcacagaaggcggccctgcccagttcgtgaacgctacaagcaaagaggtggtggaatgggccgctaagctgggcctgcccctggtgttcagatgggacgacagcaacgcccagaggaaagagtacgccggcctgtaccacgaagtggcccaggctcatggcgtggacgtgtcccaggtccggcacaagctgaccctgctggtgaaccagaacgtggacggcgaggccgctagagccgaggctagggtgtacctggaagagttcgtgcgggagagctacagcaacaccgacttcgagcagaagatgggcgagctgctgagcgagaacgccatcggcacctacgaggaaagcacccaggccgccagagtggccatcgagtgctgtggagccgccgacctgctgatgagcttcgagagcatggaagataaggcccagcagagggccgtgatcgacgtggtgcggagagtgaccgacgcccgggagaacgagatggacgagaacctggaacaggtgtccggcatcatcggcaacctgaggcacatggccctggacatgggcaacgagatcgacacccagaacaggcagatcgacaggatcatggaaaaggccgacagcaacaagaccaggatcgacgaggccaaccagagggccaccaagatgctgggaagcggcaagaagttcggcaacttcctgctgacctaccagccccctgagctgagccagaccgaagtgatgaagagactggtgaacctgggcaaggccagcgagggctgtggcttcgacaccgtgtggctgctggaacaccacttcaccgagttcggactgctgggcaacccttacgtggccgctgcccatctgctgggcgccaccgagacactgaacgtgggcaccgccgccattgtgctgcctacagcccaccctgtgcggcaggctgaggacgtgaacctgctggatcagatgtccaagggcaggttcagattcggcatctgcaggggcctgtacgacaaggacttcagggtgttcggcaccgacatggacaacagcagggccctgatggactgttggtacgacctgatgaaggaaggcttcaacgagggctacattgccgccgacaacgagcacatcaagttccctaagatccagctgaatcccagcgcctacacacagggcggagcccctgtgtacgtggtggccgagagcgcctctacaaccgagtgggctgccgagaggggcctgcccatgatcctgagctggatcatcaacacccacgagaagaaggcccagctggacctgtacaatgaggtggccaccgagcacggctacgacgtgaccaagatcgaccactgcctgagctacatcaccagcgtggaccacgactccaacagggccaaggacatctgcaggaactttctgggccattggtacgacagctacgtgaacgctaccaagatcttcgacgacagcgaccagaccaagggctacgacttcaacaagggacagtggagggacttcgtgctgaagggccacaaggacaccaacagacggatcgactacagctacgagatcaaccccgtgggcacacctgaggaatgtatcgccatcatccagcaggacatcgacgccaccggcatcgacaacatctgctgcggcttcgaggccaacggcagcgaggaagagatcattgccagcatgaagctgttccagagcgacgtgatgccctacctgaaagagaagcagggtctcagcgcttggagccacccgcagttcgaaaaataataagcttgacctgtgaagtgaaaaatggcgcacattgtgcgacattttttttgtctgccgtttaccgctactgcgtcacggatctccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattgatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaggaattaatgatgtctcgtttagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgcagtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaagcagcataacctttttccgtgatggtaacttcactagtttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgacccgaca

SEQ ID NO: 11 - DNA, 2262 bp, Artificial Sequence
  LuxBA3 with C-terminal Streptavidin-tag
atgaagttcggcctgttcttcctgaacttcatgaacagcaagaggtccagcgaccaggtgatcgaggaaatgctggacaccgcccactacgtggaccagctgaagttcgacaccctggccgtgtacgagaaccacttcagcaacaacggcgtggtgggagcccctctgacagtggccggcttcctgctgggcatgaccaagaacgccaaggtggccagcctgaaccacgtgatcaccacccaccatcctgtgcgggtggccgaggaagcctgcctgctggaccagatgagcgagggcagattcgccttcggcttcagcgactgcgagaagtccgccgacatgcggttcttcaacaggcccaccgacagccagttccagctgttcagcgagtgccacaagatcatcaacgacgccttcaccaccggctactgccaccccaacaacgacttctacagcttccccaagatcagcgtgaacccccacgccttcacagaaggcggccctgcccagttcgtgaacgctacaagcaaagaggtggtggaatgggccgctaagctgggcctgcccctggtgttcagatgggacgacagcaacgcccagaggaaagagtacgccggcctgtaccacgaagtggcccaggctcatggcgtggacgtgtcccaggtccggcacaagctgaccctgctggtgaaccagaacgtggacggcgaggccgctagagccgaggctagggtgtacctggaagagttcgtgcgggagagctacagcaacaccgacttcgagcagaagatgggcgagctgctgagcgagaacgccatcggcacctacgaggaaagcacccaggccgccagagtggccatcgagtgctgtggagccgccgacctgctgatgagcttcgagagcatggaagataaggcccagcagagggccgtgatcgacgtggtgcggagagtgaccgacgcccgggagaacgagatggacgagaacctggaacaggtgtccggcatcatcggcaacctgaggcacatggccctggacatgggcaacgagatcgacacccagaacaggcagatcgacaggatcatggaaaaggccgacagcaacaagaccaggatcgacgaggccaaccagagggccaccaagatgctgggaagcggcaagaagttcggcaacttcctgctgacctaccagccccctgagctgagccagaccgaagtgatgaagagactggtgaacctgggcaaggccagcgagggctgtggcttcgacaccgtgtggctgctggaacaccacttcaccgagttcggactgctgggcaacccttacgtggccgctgcccatctgctgggcgccaccgagacactgaacgtgggcaccgccgccattgtgctgcctacagcccaccctgtgcggcaggctgaggacgtgaacctgctggatcagatgtccaagggcaggttcagattcggcatctgcaggggcctgtacgacaaggacttcagggtgttcggcaccgacatggacaacagcagggccctgatggactgttggtacgacctgatgaaggaaggcttcaacgagggctacattgccgccgacaacgagcacatcaagttccctaagatccagctgaatcccagcgcctacacacagggcggagcccctgtgtacgtggtggccgagagcgcctctacaaccgagtgggctgccgagaggggcctgcccatgatcctgagctggatcatcaacacccacgagaagaaggcccagctggacctgtacaatgaggtggccaccgagcacggctacgacgtgaccaagatcgaccactgcctgagctacatcaccagcgtggaccacgactccaacagggccaaggacatctgcaggaactttctgggccattggtacgacagctacgtgaacgctaccaagatcttcgacgacagcgaccagaccaagggctacgacttcaacaagggacagtggagggacttcgtgctgaagggccacaaggacaccaacagacggatcgactacagctacgagatcaaccccgtgggcacacctgaggaatgtatcgccatcatccagcaggacatcgacgccaccggcatcgacaacatctgctgcggcttcgaggccaacggcagcgaggaagagatcattgccagcatgaagctgttccagagcgacgtgatgccctacctgaaagagaagcagggtctcagcgcttggagccacccgcagttcgaaaaataa

SEQ ID NO: 12 - DNA, 2937 bp, Artificial Sequence
  LuxBA3 with N-terminal glutathione-S-transferase and C-terminal Streptavidin-tag
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctgatcgaaggtcggatgaagttcggcctgttcttcctgaacttcatgaacagcaagaggtccagcgaccaggtgatcgaggaaatgctggacaccgcccactacgtggaccagctgaagttcgacaccctggccgtgtacgagaaccacttcagcaacaacggcgtggtgggagcccctctgacagtggccggcttcctgctgggcatgaccaagaacgccaaggtggccagcctgaaccacgtgatcaccacccaccatcctgtgcgggtggccgaggaagcctgcctgctggaccagatgagcgagggcagattcgccttcggcttcagcgactgcgagaagtccgccgacatgcggttcttcaacaggcccaccgacagccagttccagctgttcagcgagtgccacaagatcatcaacgacgccttcaccaccggctactgccaccccaacaacgacttctacagcttccccaagatcagcgtgaacccccacgccttcacagaaggcggccctgcccagttcgtgaacgctacaagcaaagaggtggtggaatgggccgctaagctgggcctgcccctggtgttcagatgggacgacagcaacgcccagaggaaagagtacgccggcctgtaccacgaagtggcccaggctcatggcgtggacgtgtcccaggtccggcacaagctgaccctgctggtgaaccagaacgtggacggcgaggccgctagagccgaggctagggtgtacctggaagagttcgtgcgggagagctacagcaacaccgacttcgagcagaagatgggcgagctgctgagcgagaacgccatcggcacctacgaggaaagcacccaggccgccagagtggccatcgagtgctgtggagccgccgacctgctgatgagcttcgagagcatggaagataaggcccagcagagggccgtgatcgacgtggtgcggagagtgaccgacgcccgggagaacgagatggacgagaacctggaacaggtgtccggcatcatcggcaacctgaggcacatggccctggacatgggcaacgagatcgacacccagaacaggcagatcgacaggatcatggaaaaggccgacagcaacaagaccaggatcgacgaggccaaccagagggccaccaagatgctgggaagcggcaagaagttcggcaacttcctgctgacctaccagccccctgagctgagccagaccgaagtgatgaagagactggtgaacctgggcaaggccagcgagggctgtggcttcgacaccgtgtggctgctggaacaccacttcaccgagttcggactgctgggcaacccttacgtggccgctgcccatctgctgggcgccaccgagacactgaacgtgggcaccgccgccattgtgctgcctacagcccaccctgtgcggcaggctgaggacgtgaacctgctggatcagatgtccaagggcaggttcagattcggcatctgcaggggcctgtacgacaaggacttcagggtgttcggcaccgacatggacaacagcagggccctgatggactgttggtacgacctgatgaaggaaggcttcaacgagggctacattgccgccgacaacgagcacatcaagttccctaagatccagctgaatcccagcgcctacacacagggcggagcccctgtgtacgtggtggccgagagcgcctctacaaccgagtgggctgccgagaggggcctgcccatgatcctgagctggatcatcaacacccacgagaagaaggcccagctggacctgtacaatgaggtggccaccgagcacggctacgacgtgaccaagatcgaccactgcctgagctacatcaccagcgtggaccacgactccaacagggccaaggacatctgcaggaactttctgggccattggtacgacagctacgtgaacgctaccaagatcttcgacgacagcgaccagaccaagggctacgacttcaacaagggacagtggagggacttcgtgctgaagggccacaaggacaccaacagacggatcgactacagctacgagatcaaccccgtgggcacacctgaggaatgtatcgccatcatccagcaggacatcgacgccaccggcatcgacaacatctgctgcggcttcgaggccaacggcagcgaggaagagatcattgccagcatgaagctgttccagagcgacgtgatgccctacctgaaagagaagcagggtctcagcgcttggagccacccgcagttcgaaaaataa

SEQ ID NO: 13 - DNA, 2307 bp, Artificial Sequence
  LuxBA3 with N-terminal 9xHistidin and C-terminal Streptavidin-tag
atgggccatcatcatcatcatcaccaccatcacatcgaaggtcggatgaagttcggcctgttcttcctgaacttcatgaacagcaagaggtccagcgaccaggtgatcgaggaaatgctggacaccgcccactacgtggaccagctgaagttcgacaccctggccgtgtacgagaaccacttcagcaacaacggcgtggtgggagcccctctgacagtggccggcttcctgctgggcatgaccaagaacgccaaggtggccagcctgaaccacgtgatcaccacccaccatcctgtgcgggtggccgaggaagcctgcctgctggaccagatgagcgagggcagattcgccttcggcttcagcgactgcgagaagtccgccgacatgcggttcttcaacaggcccaccgacagccagttccagctgttcagcgagtgccacaagatcatcaacgacgccttcaccaccggctactgccaccccaacaacgacttctacagcttccccaagatcagcgtgaacccccacgccttcacagaaggcggccctgcccagttcgtgaacgctacaagcaaagaggtggtggaatgggccgctaagctgggcctgcccctggtgttcagatgggacgacagcaacgcccagaggaaagagtacgccggcctgtaccacgaagtggcccaggctcatggcgtggacgtgtcccaggtccggcacaagctgaccctgctggtgaaccagaacgtggacggcgaggccgctagagccgaggctagggtgtacctggaagagttcgtgcgggagagctacagcaacaccgacttcgagcagaagatgggcgagctgctgagcgagaacgccatcggcacctacgaggaaagcacccaggccgccagagtggccatcgagtgctgtggagccgccgacctgctgatgagcttcgagagcatggaagataaggcccagcagagggccgtgatcgacgtggtgcggagagtgaccgacgcccgggagaacgagatggacgagaacctggaacaggtgtccggcatcatcggcaacctgaggcacatggccctggacatgggcaacgagatcgacacccagaacaggcagatcgacaggatcatggaaaaggccgacagcaacaagaccaggatcgacgaggccaaccagagggccaccaagatgctgggaagcggcaagaagttcggcaacttcctgctgacctaccagccccctgagctgagccagaccgaagtgatgaagagactggtgaacctgggcaaggccagcgagggctgtggcttcgacaccgtgtggctgctggaacaccacttcaccgagttcggactgctgggcaacccttacgtggccgctgcccatctgctgggcgccaccgagacactgaacgtgggcaccgccgccattgtgctgcctacagcccaccctgtgcggcaggctgaggacgtgaacctgctggatcagatgtccaagggcaggttcagattcggcatctgcaggggcctgtacgacaaggacttcagggtgttcggcaccgacatggacaacagcagggccctgatggactgttggtacgacctgatgaaggaaggcttcaacgagggctacattgccgccgacaacgagcacatcaagttccctaagatccagctgaatcccagcgcctacacacagggcggagcccctgtgtacgtggtggccgagagcgcctctacaaccgagtgggctgccgagaggggcctgcccatgatcctgagctggatcatcaacacccacgagaagaaggcccagctggacctgtacaatgaggtggccaccgagcacggctacgacgtgaccaagatcgaccactgcctgagctacatcaccagcgtggaccacgactccaacagggccaaggacatctgcaggaactttctgggccattggtacgacagctacgtgaacgctaccaagatcttcgacgacagcgaccagaccaagggctacgacttcaacaagggacagtggagggacttcgtgctgaagggccacaaggacaccaacagacggatcgactacagctacgagatcaaccccgtgggcacacctgaggaatgtatcgccatcatccagcaggacatcgacgccaccggcatcgacaacatctgctgcggcttcgaggccaacggcagcgaggaagagatcattgccagcatgaagctgttccagagcgacgtgatgccctacctgaaagagaagcagggtctcagcgcttggagccacccgcagttcgaaaaataa

SEQ ID NO: 14 - DNA, 7555 bp, Artificial Sequence
  ligation of LuxBA3 into pTZ_E47 vector
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggccatcatcatcatcatcaccaccatcacatcgaaggtcggatgaagttcggcctgttcttcctgaacttcatgaacagcaagaggtccagcgaccaggtgatcgaggaaatgctggacaccgcccactacgtggaccagctgaagttcgacaccctggccgtgtacgagaaccacttcagcaacaacggcgtggtgggagcccctctgacagtggccggcttcctgctgggcatgaccaagaacgccaaggtggccagcctgaaccacgtgatcaccacccaccatcctgtgcgggtggccgaggaagcctgcctgctggaccagatgagcgagggcagattcgccttcggcttcagcgactgcgagaagtccgccgacatgcggttcttcaacaggcccaccgacagccagttccagctgttcagcgagtgccacaagatcatcaacgacgccttcaccaccggctactgccaccccaacaacgacttctacagcttccccaagatcagcgtgaacccccacgccttcacagaaggcggccctgcccagttcgtgaacgctacaagcaaagaggtggtggaatgggccgctaagctgggcctgcccctggtgttcagatgggacgacagcaacgcccagaggaaagagtacgccggcctgtaccacgaagtggcccaggctcatggcgtggacgtgtcccaggtccggcacaagctgaccctgctggtgaaccagaacgtggacggcgaggccgctagagccgaggctagggtgtacctggaagagttcgtgcgggagagctacagcaacaccgacttcgagcagaagatgggcgagctgctgagcgagaacgccatcggcacctacgaggaaagcacccaggccgccagagtggccatcgagtgctgtggagccgccgacctgctgatgagcttcgagagcatggaagataaggcccagcagagggccgtgatcgacgtggtgcggagagtgaccgacgcccgggagaacgagatggacgagaacctggaacaggtgtccggcatcatcggcaacctgaggcacatggccctggacatgggcaacgagatcgacacccagaacaggcagatcgacaggatcatggaaaaggccgacagcaacaagaccaggatcgacgaggccaaccagagggccaccaagatgctgggaagcggcaagaagttcggcaacttcctgctgacctaccagccccctgagctgagccagaccgaagtgatgaagagactggtgaacctgggcaaggccagcgagggctgtggcttcgacaccgtgtggctgctggaacaccacttcaccgagttcggactgctgggcaacccttacgtggccgctgcccatctgctgggcgccaccgagacactgaacgtgggcaccgccgccattgtgctgcctacagcccaccctgtgcggcaggctgaggacgtgaacctgctggatcagatgtccaagggcaggttcagattcggcatctgcaggggcctgtacgacaaggacttcagggtgttcggcaccgacatggacaacagcagggccctgatggactgttggtacgacctgatgaaggaaggcttcaacgagggctacattgccgccgacaacgagcacatcaagttccctaagatccagctgaatcccagcgcctacacacagggcggagcccctgtgtacgtggtggccgagagcgcctctacaaccgagtgggctgccgagaggggcctgcccatgatcctgagctggatcatcaacacccacgagaagaaggcccagctggacctgtacaatgaggtggccaccgagcacggctacgacgtgaccaagatcgaccactgcctgagctacatcaccagcgtggaccacgactccaacagggccaaggacatctgcaggaactttctgggccattggtacgacagctacgtgaacgctaccaagatcttcgacgacagcgaccagaccaagggctacgacttcaacaagggacagtggagggacttcgtgctgaagggccacaaggacaccaacagacggatcgactacagctacgagatcaaccccgtgggcacacctgaggaatgtatcgccatcatccagcaggacatcgacgccaccggcatcgacaacatctgctgcggcttcgaggccaacggcagcgaggaagagatcattgccagcatgaagctgttccagagcgacgtgatgccctacctgaaagagaagcagggtctcagcgcttggagccacccgcagttcgaaaaataataggcgcgccggatcctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

(19) United States
(12) Patent Application Publication
(10) Pub. No.: US 2015/0044709 A1
(43) Pub. Date: Feb. 12, 2015

(54) MEANS AND METHODS FOR DETERMINING NEUROTOXIN ACTIVITY BASED ON A MODIFIED LUCIFERASE

(71) Applicant: MERZ PHARMA GmbH & CO KGaA, FRANKFURT am MAIN (DE)

(72) Inventor: Karl-Heinz Eisele, Frankfurt Am Main (DE)

(21) Appl. No.: 14/382,929

(22) PCT Filed: Mar. 7, 2013
(86) PCT No.: PCT/EP2013/054566

§ 371 Date: Sep. 4, 2014

Related U.S. Application Data
(60) Provisional application No. 61/607,760, filed on Mar. 7, 2012.

(30) Foreign Application Priority Data
Mar. 7, 2012 (EP) 12158450.2

(51) Int. Cl. C12Q 1/66; C12N 9/02

(52) U.S. Cl. CPC C12Q 1/66; C12N 9/0069; C07K 2319/50; G01N 2333/952; C12Y 113/12
USPC 435/8; 536/23.2; 435/320.1; 435/325; 435/189

(57) ABSTRACT

The present invention is concerned with test systems for determining the activity of neurotoxin polypeptides. Specifically, it relates to a polynucleotide encoding a single chain luciferase fusion polypeptide comprising: (i) a LuxB subunit, (ii) a linker comprising a neurotoxin cleavage site, and (iii) a LuxA subunit and a polypeptide encoded by the polynucleotide. Further provided in accordance with the invention are a vector and a host cell comprising the polynucleotide. Moreover, the present invention relates to a method for determining a proteolytically active neurotoxin polypeptide in a sample and a kit for carrying out the method.

The present invention is concerned with test systems for determining the activity of neurotoxin polypeptides. Specifically, it relates to a polynucleotide encoding a single chain luciferase fusion polypeptide comprising: (i) a LuxB subunit, (ii) a linker comprising a neurotoxin cleavage site, and (iii) a LuxA subunit and a polypeptide encoded by said polynucleotide. Further provided in accordance with the invention are a vector and a host cell comprising said polynucleotide. Moreover, the present invention relates to a method for determining a proteolytically active neurotoxin polypeptide in a sample and a kit for carrying out said method.

Clostridium botulinum and Clostridium tetani produce highly potent neurotoxins, i.e. botulinum toxins (BoNTs) and tetanus toxin (TeNT), respectively. These Clostridial neurotoxins specifically bind to neuronal cells and disrupt neurotransmitter release. Each toxin is synthesized as an inactive unprocessed approximately 150 kDa single-chain protein. The posttranslational processing involves formation of disulfide bridges, and limited proteolysis (nicking) by bacterial protease(s). Active dichain neurotoxin consists of two chains, an N-terminal light chain of approx. 50 kDa and a heavy chain of approx. 100 kDa linked by a disulfide bond. Neurotoxins structurally consist of three domains, i.e. the catalytic light chain, the heavy chain encompassing the translocation domain (N-terminal half) and the receptor binding domain (C-terminal half), see Krieglstein 1990, Eur J Biochem 188, 39; Krieglstein 1991, Eur J Biochem 202, 41; Krieglstein 1994, J Protein Chem 13, 49.

Clostridium botulinum secretes seven antigenically distinct serotypes designated A to G of the BoNTs. All serotypes together with the related TeNT secreted by Clostridium tetani, are zinc (Zn2+)-dependent endoproteases that block synaptic exocytosis by cleaving SNARE proteins and, in particular in the case of BoNT/A, C or E, SNAP-25. BoNTs cause, inter alia, the flaccid muscular paralysis seen in botulism, see Fischer 2007, PNAS 104, 10447.

Despite its toxic effects, BoNTs have been used as a therapeutic agents in a large number of diseases. BoNT serotype A (BoNT/A) was approved for human use in the United States in 1989 for the treatment of strabism, blepharospasm, and other disorders. It is commercially available as a protein preparation, for example, under the tradename BOTOX (Allergan Inc) under the tradename DYSPORT (Ipsen Ltd). For therapeutic application the complex is injected directly into the muscle to be treated. At physiological pH, the toxin is released from the protein complex and the desired pharmacological effect takes place. An improved BoNT/A preparation being free of complexing proteins is available under the tradename XEOMIN (Merz Pharmaceuticals GmbH).

BoNTs, in principle, weaken voluntary muscle strength and are, therefore, effective therapeutic agents for the therapy of diseases such as strabism, focal dystonia, including cervical dystonia, and benign essential blepharospasm or spasticity. They have been further shown to relief hemifacial spasm, and focal spasticity, and moreover, to be effective in a wide range of other indications, such as gastrointestinal disorders, hyperhidrosis, and cosmetic wrinkle correction, see Jost 2007, Drugs 67, 669.

The determination of the biological activity is important as a safety measure, for quality control and for quantification purposes. The mouse LD50 assay is currently the only reliable assay for quantifying the biological activity of neurotoxins and for assessing their therapeutic potential and/or their toxicity. Said assay is also accepted for quality control purposes during manufacture of neurotoxin. In the mouse LD50 bioassay, lethal and sub-lethal concentrations of a sample containing the neurotoxin polypeptide have to be injected into at least 120 animals. The number of killed animals over an observation period of 72 hours allows determining the neurotoxin polypeptide concentration in the sample. Apparent drawbacks of this assay are the high number of animals which will be sacrificed and the high level of stress and pain for said animals during the test.

In vitro assays which have been proposed so far are based on determining SNAP-25 cleavage in a cell free system or on neurotoxin exposure to primary neurons. However, these assay are less reliable and/or do not take into account all of the desired neurotoxin functions. Thus, at present, the LD50 bioassay described above is the only reliable assay which is described in the monograph for BoNT/A in the European pharmacopeia. However, there is a need for a reliable assay for measuring neurotoxin activity which avoids the drawbacks of the LD50 bioassay.

Therefore, the technical problem underlying the present invention could be seen in the provision of means and methods for complying with the aforementioned needs. The technical problem is solved by the embodiments characterized in the claims and herein described below.

The present invention relates to a polynucleotide encoding a single chain luciferase fusion polypeptide comprising: (i) a LuxB subunit, (ii) a linker comprising a neurotoxin cleavage site, and (iii) a LuxA subunit.

The term “polynucleotide” as used herein refers to single- or double-stranded DNA molecules as well as to RNA molecules. Encompassed by the said term is genomic DNA, cDNA, hnRNA, mRNA as well as all naturally occurring or artificially modified derivatives of such molecular species. The polynucleotide may be in an aspect a linear or circular molecule. Moreover, in addition to the nucleic acid sequences encoding the polypeptide of the present invention, a polynucleotide of the present invention may comprise additional sequences required for proper transcription and/or translation such as 5′- or 3′-UTR sequences. The nucleic acid sequences encoding the polypeptide of the present invention can be derived from the amino acid sequence envisaged for the single chain luciferase fusion polypeptide of the invention by a skilled artisan without further ado. In light of the degeneracy of the genetic code, optimized codons may be used in the nucleic acid sequences encoding the single chain luciferase fusion polypeptide in the polynucleotide of the present invention. Thereby, optimal expression in, e.g., a host cell of the present invention can be achieved.

The term “single chain luciferase fusion polypeptide” as used herein refers to a polypeptide comprising within a single polypeptide chain a LuxB subunit of a luciferase as well as a LuxA subunit. The said subunits are separated by a linker that comprises a neurotoxin cleavage site which is specifically recognized and cleaved by a proteolytically active neurotoxin polypeptide as referred to elsewhere herein. The single chain luciferase fusion polypeptide, in an aspect, has the LuxB and LuxA subunits arranged in a manner which prevents or reduces the formation of a biologically active luciferase holoenzyme when the subunits are present in the single chain luciferase fusion polypeptide. In an aspect, a biologically active luciferase holoenzyme is, thus, formed only or formed more efficiently after cleavage of the single chain luciferase fusion polypeptide by the neurotoxin protease. If the enzymatic activity, in an aspect, is prevented, essentially no activity shall occur when the single chain luciferase fusion polypeptide is in the single chain state. A reduced enzymatic activity as referred to herein, in an aspect, means a statistically significant reduced activity. In an aspect, the reduction of the activity in the single chain state versus the holoenzyme formed after cleavage is at least 10%, at least 20%, at least 30%, at least 40%, at least 50%, at least 60%, at least 70% or at least 80%.

In an aspect, the LuxB and LuxA subunits in the single chain luciferase fusion polypeptide are arranged in a manner which prevents or reduces the formation of a biologically active luciferase holoenzyme in that the order of the subunits in the single chain luciferase fusion polypeptide is from N to C-terminus: (i) a LuxB subunit, (ii) a linker comprising a neurotoxin cleavage site, and (iii) a LuxA subunit. Such an order of the subunits has been described to efficiently prevent luciferase activity in a single chain fusion protein (see Olsson 1989, Gene 81: 335-347).

In another aspect, the LuxB and LuxA subunits in the single chain luciferase fusion polypeptide are separated in addition to the linker by further polypeptide elements which prevent or reduce the capability of the subunits to interact with each other. This can be achieved, in an aspect, by inserting ankyrin repeats into the single chain luciferase fusion polypeptide either between the LuxB subunit and the linker or between the LuxA subunit and the linker or both. Ankyrin repeats are known to reduce the flexibility of a polypeptide chain and, thus, will reduce the capability of the separated luciferase subunits in the single chain luciferase fusion polypeptide of the invention to interact with each other. Ankyrins are well known in the art and belong into the family of adaptor proteins mediating the attachment of transmembrane proteins and cytoskeletal proteins. In mammals, three different ankyrins are known, ANK1, ANK2 and ANK3, occurring in various splice forms. The ankyrins have an N-terminal domain comprising the so-called ankyrin repeats which are to be applied in accordance with the present invention (see Wetzel 2008, J Mol Biol. 376(1): 241-57; Michaely 1995, J Biol Chem. 270(52):31298-302; Batrukova 2000, Biochemistry (Mosc). 65(4):395-408.).

In another aspect, a prevention or reduction of the capability of the subunits to interact with each other can be achieved by inserting protein domains or proteins either between the LuxB subunit and the linker, between the LuxA subunit and the linker or both which due to their molecular size sterically interfere with the interaction of the subunits with each other. In an aspect, globular proteins or domains thereof can be applied for this purpose. In an aspect, the said globular protein or domain thereof is selected from the group consisting: glutathione S-transferase (GST), maltose binding protein, small ubiquitin-like modifier (SUMO) protein, green fluorescent protein (GFP), red fluorescent protein (RFP), yellow fluorescent protein (YFP), blue fluorescent protein (BFP), and the like. In another aspect, a fluorescent protein may be bound to the neurotoxin and/or luciferase polypeptides specified elsewhere herein, whereby said fluorescent protein e.g. alters light emission of said polypeptide and/or is be used as internal control to monitor expression, number of cells, loading control, and the like. In yet an aspect, the linker can be comprised in a globular protein or domain thereof as described before.

It will be understood that the neurotoxin cleavage site comprised in the linker shall be made available by the single chain luciferase fusion protein to a neurotoxin polypeptide such that the neurotoxin protease can recognize, bind to and cleave the single chain luciferase fusion polypeptide under suitable conditions. The skilled artisan is well aware of how a suitable arrangement within the single chain luciferase fusion polypeptide can be designed. Moreover, the single chain luciferase fusion protein can be tested for cleavage by proteolytically active neurotoxin polypeptide as described in the accompanying Examples. In an aspect, the single chain luciferase fusion polypeptide of the invention has an amino acid sequence as shown in any one of SEQ ID NOs: 1 to 3.

The term “luciferase” as used herein refers to enzymes belonging into a class of enzymes capable of catalyzing a light-emitting reaction. Luciferases occur naturally as firely or bacterial luciferases. Such luciferases as enzymatically (biologically) active holoenzymes are composed of different subunits. In an aspect, the luciferase referred to herein is a bacterial luciferase and the subunits to be inserted into the single chain luciferase fusion polypeptide are LuxA and LuxB. In yet another aspect, the said LuxA and/or LuxB subunits are from Vibrio fischeri or Vibrio harveyi. How to arrange such subunits in a singly chain luciferase fusion protein without additional linker according to the present invention is known in the art and described in Olsson 1989, loc cit. The structure of the aforementioned luciferases and their subunits as well as nucleic acid sequences encoding them are well known in the art and describe, e.g., in Chon 1985, J Biol Chem. 260(10):6139-46 and Johnston 1986, J Biol Chem. 261(11):4805-11. In an aspect, the LuxA subunit of Vibrio fischeri as referred to herein has an amino acid sequence as shown in SEQ ID NO: 4 or a variant thereof. In another aspect, the LuxB subunit of Vibrio fischeri has an amino acid sequence as shown in SEQ ID NO: 5 or a variant thereof. It will be understood that the present invention also encompasses variants of such specific amino acid or nucleic acid sequences encoding them as long as these variant sequences also allow for the formation of an enzymatically active luciferase holoenzyme. In an aspect, a sequence variant as used herein differs from the specific amino acid sequence or a specific nucleic acid sequence as specified before by one or more amino acid or nucleotide substitutions, additions and/or deletions. In another aspect, the said variant sequence is at least 50%, at least 60%, at least 70%, at least 80%, at least 90%, at least 95%, at least 98% or at least 99% identical to the specific sequence over the entire length or over at least a stretch of half of the length of the specific sequence. The term “identical” as used herein refers to sequence identity characterized by determining the number of identical amino acids between sequences wherein the sequences are aligned so that the highest order match is obtained. It can be calculated using published techniques or methods codified in computer programs such as, for example, BLASTP or FASTA (Altschul 1990, J Mol Biol 215, 403). The percent identity values are, in one aspect, calculated over the entire amino acid sequence or over a sequence stretch of at least 50% of the query sequence. A series of programs based on a variety of algorithms is available to the skilled worker for comparing different sequences. In this context, the algorithms of Needleman and Wunsch or Smith and Waterman give particularly reliable results. To carry out the sequence alignments, the program PileUp (Higgins 1989, CABIOS 5, 151) or the programs Gap and BestFit (Needleman 1970, J Mol Biol 48; 443; Smith 1981, Adv Appl Math 2, 482), which are part of the GCG software packet (Genetics Computer Group 1991, 575 Science Drive, Madison, Wis., USA 53711), may be used. The sequence identity values recited above in percent (%) are to be determined, in another aspect of the invention, using the program GAP over the entire sequence region with the following settings: Gap Weight: 50, Length Weight: 3, Average Match: 10.000 and Average Mismatch: 0.000, which, unless otherwise specified, shall always be used as standard settings for sequence alignments.

The term “neurotoxin cleavage site” as used herein refers to a cleavage site which is recognized and cleaved by the endogenous protease of a neurotoxin polypeptide. Cleavage sites which are recognized by the neurotoxin proteases are well known in the art (see, e.g., EP 1 926 744 B1). In an aspect, the said neurotoxin cleavage site is selected from the group consisting of: a neurotoxin cleavage site from SNAP-25, a neurotoxin cleavage site from VAMP, a neurotoxin cleavage site from syntaxin, and the autocatalytic cleavage sites from neurotoxin polypeptides.

In principle, a neurotoxin cleavage site can be a cleavage site which naturally occurs in a substrate or which is an artificially designed cleavage site recognized and cleaved by the neurotoxin polypeptides protease. It will be understood that the properties of the neurotoxin cleavage site govern the kind of neurotoxin which can activate the polypeptide of the present invention. Neurotoxin polypeptides referred to herein, in an aspect, encompass BoNT/A, BoNT/B, BoNT/C1, BoNT/D, BoNT/E, BoNT/G, BoNT/F or TeNT all of which are well known in the art. For example, if a neurotoxin cleavage site is used which is specifically recognized and cleaved by BoNT/A, only the BoNT/A protease will be capable of activating the polypeptide of the present invention and, in particular, its luciferase activity, whereas if a neurotoxin cleavage site is used which is specifically recognized and cleaved by BoNT/E, only the BoNT/E protease will be capable of activating the polypeptide of the present invention and, in particular, its caspase activity. In an aspect of the invention, the neurotoxin cleavage site is cleaved by mature BoNTs. In yet another aspect, it is cleaved by muteins of BoNTs, in an aspect, by muteins comprising or consisting of the BoNT light chain exhibiting the BoNT protease activity.

A neurotoxin cleavage site recognized and cleaved by the BoNT/A protease, in an aspect of the invention, is derived from a protein that is sensitive to cleavage by BoNT/A. In an aspect, such a protein is human SNAP-25A or -25B or a homolog, paralog or ortholog thereof from rat, mouse, bovine, Danio, Carassius, Xenopus, Torpedo, Strongylocentrotus, Loligo, Lymnaea or Aplysia. Suitable cleavage sites derived from said proteins are disclosed in EP 1 926 744 B1.

A neurotoxin cleavage site recognized and cleaved by the BoNT/B protease, in an aspect of the invention, is derived from a protein that is sensitive to cleavage by BoNT/B. In an aspect, such a protein is human or mouse VAMP-1, VAMP-2 and VAMP-3/cellubrevin, bovine VAMP-2, rat VAMP-2 or VAMP-3, chicken VAMP-1, VAMP-2 or VAMP-3, Torpedo VAMP-1, Strongylocentrotus VAMP, Drosophila sybA, synB, synC, synD, or syn, Hirudo VAMP, Xenopus VAMP-2 or VAMP-3, Danio VAMP-1 or VAMP-2, Loligo VAMP, Lymnaea VAMP, Aplysia VAMP or Caenorhabditis SNB1-like or any ortholog, paralog or homolog thereof. Suitable cleavage sites derived from said proteins are disclosed in EP 1 926 744 B1.

A neurotoxin cleavage site recognized and cleaved by the BoNT/C1 protease, in an aspect of the invention, is derived from a protein that is sensitive to cleavage by BoNT/C1. In an aspect, such a protein is human and mouse Syntaxin 1A, Syntaxin 1B1, Syntaxin 2-1, Syntaxin 2-2, Syntaxin 2-3, Syntaxin 3A or Syntaxin 1B2, bovine or rat Syntaxin 1A, Syntaxin 1B1 or Syntaxin 1B2, rat Syntaxin 2 or Rat syntaxin 3, mouse Syntaxin 1A, Syntaxin 1B1, Syntaxin 1B2, Syntaxin 2, Syntaxin 3A, Syntaxin 3B or Syntaxin 3C, chicken Syntaxin 1A or Syntaxin 2; Xenopus Syntaxin 1A or Syntaxin 1B, Danio Syntaxin 1A, Syntaxin 1B or Syntaxin 3, Torpedo Syntaxin 1A or Syntaxin 1B, Strongylocentrotus Syntaxin 1A or Syntaxin 1B, Drosophila Syntaxin 1A or Syntaxin 1B, Hirudo Syntaxin 1A or Syntaxin 1B, Loligo Syntaxin 1A or Syntaxin 1B, Lymnaea Syntaxin 1A or Syntaxin 1B or any ortholog, paralog or homolog thereof. Suitable cleavage sites derived from said proteins are disclosed in EP 1 926 744 B1.

A neurotoxin cleavage site recognized and cleaved by the BoNT/D protease, in an aspect of the invention, is derived from a protein that is sensitive to cleavage by BoNT/D. In an aspect, such a protein is human or mouse VAMP-1, VAMP-2 and VAMP-3/cellubrevin, bovine VAMP-2, rat VAMP-2 or VAMP-3, chicken VAMP-1, VAMP-2 or VAMP-3, Torpedo VAMP-1, Strongylocentrotus VAMP, Drosophila sybA, synB, synC, synD, or syn, Hirudo VAMP, Xenopus VAMP-2 or VAMP-3, Danio VAMP-1 or VAMP-2, Loligo VAMP, Lymnaea VAMP, Aplysia VAMP or Caenorhabditis SNB1-like or any ortholog, paralog or homolog thereof. Suitable cleavage sites derived from said proteins are disclosed in EP 1 926 744 B1.

A neurotoxin cleavage site recognized and cleaved by the BoNT/E protease, in an aspect of the invention, is derived from a protein that is sensitive to cleavage by BoNT/E. In an aspect, such a protein is, such a protein is human SNAP-25A or B or a homolog, paralog or ortholog thereof from rat, mouse, bovine, Danio, Carassius, Xenopus, Torpedo, Strongylocentrotus, Loligo, Lymnaea or Aplysia. Suitable cleavage sites derived from said proteins are disclosed in EP 1 926 744 B1.

A neurotoxin cleavage site recognized and cleaved by the BoNT/F protease, in an aspect of the invention, is derived from a protein that is sensitive to cleavage by BoNT/F. In an aspect, such a protein is, such a protein is human or mouse VAMP-1, VAMP-2 and VAMP-3/cellubrevin, bovine VAMP-2, rat VAMP-2 or VAMP-3, chicken VAMP-1, VAMP-2 or VAMP-3, Torpedo VAMP-1, Strongylocentrotus VAMP, Drosophila sybA, synB, synC, synD, or syn, Hirudo VAMP, Xenopus VAMP-2 or VAMP-3, Danio VAMP-1 or VAMP-2, Loligo VAMP, Lymnaea VAMP, Aplysia VAMP or Caenorhabditis SNB1-like or any ortholog, paralog or homolog thereof. Suitable cleavage sites derived from said proteins are disclosed in EP 1 926 744 B1.

A neurotoxin cleavage site recognized and cleaved by the BoNT/G protease, in an aspect of the invention, is derived from a protein that is sensitive to cleavage by BoNT/G. In an aspect, such a protein is, such a protein is human or mouse VAMP-1, VAMP-2 and VAMP-3/cellubrevin, bovine VAMP-2, rat VAMP-2 or VAMP-3, chicken VAMP-1, VAMP-2 or VAMP-3, Torpedo VAMP-1, Strongylocentrotus VAMP, Drosophila sybA, synB, synC, synD, or syn, Hirudo VAMP, Xenopus VAMP-2 or VAMP-3, Danio VAMP-1 or VAMP-2, Loligo VAMP, Lymnaea VAMP, Aplysia VAMP or Caenorhabditis SNB1-like or any ortholog, paralog or homolog thereof. Suitable cleavage sites derived from said proteins are disclosed in EP 1 926 744 B1.

A neurotoxin cleavage site recognized and cleaved by the TeNT protease, in an aspect of the invention, is derived from a protein that is sensitive to cleavage by TeNT. In an aspect, such a protein is human or mouse VAMP-1, VAMP-2 and VAMP-3/cellubrevin, bovine VAMP-2, rat VAMP-2 or VAMP-3, chicken VAMP-1, VAMP-2 or VAMP-3, Torpedo VAMP-1, Strongylocentrotus VAMP, Drosophila sybA, synB, synC, synD, or syn, Hirudo VAMP, Xenopus VAMP-2 or VAMP-3, Danio VAMP-1 or VAMP-2, Loligo VAMP, Lymnaea VAMP, Aplysia VAMP or Caenorhabditis SNB1-like or any ortholog, paralog or homolog thereof. Suitable cleavage sites derived from said proteins are disclosed in EP 1 926 744 B1.

A neurotoxin cleavage site recognized and cleaved by the BoNT proteases, in another aspect of the invention, is derived from the autocatalytic cleavage sites found in the BoNT proteins. In aspects, a neurotoxin cleavage site to be used in accordance with the present invention and which is derived from the autocatalytic cleavage site of a given BoNT or TeNT comprises at least 6, at least 8, at least 10 or at least 15 consecutive residues of including the BoNT/A residues 250Tyr-251Tyr, the BoNT/B residues 256Phe-257Phe, the BoNT/C1 residues 257Phe-258Tyr, the BoNT/D residues 257Phe-258Phe, the BoNT/E residues 239Pro-240Leu, the BoNT/F residues 254Pro-255Leu, the BoNT/G residues 256Phe-257Phe, the TeNT residues 259Ile-260Tyr, the BoNT/A residues Phe266-Gly267, the BoNT/B residues Phe272-Gly273, the BoNT/C1 residues Phe273-Gly274, the BoNT/D residues Phe273-Gly274, the BoNT/E residues Phe255-Gly256, the BoNT/F residues Phe270-Gly271, the BoNT/G residues Phe272-Gly273 or the TeNT residues Phe275-Gly276. Suitable cleavage sites derived from said BoNTs and TeNT are disclosed in EP 1 926 744 B1.

In yet an aspect of the invention, said neurotoxin cleavage site is a SNAP-25 derived cleavage site as shown in any one of SEQ ID NOs: 6 to 8.

Advantageously, the single chain luciferase fusion polypeptide of the present invention allows for the determination of the qualitative or quantitative protease activity of a given neurotoxin polypeptide in a cell culture system or in a cell free assay. Thus, expensive and unnecessary animal testing can be avoided or reduced thanks to the present invention. Moreover, the single chain luciferase fusion polypeptide can be applied in automated high throughput screening assays. The single chain luciferase fusion polypeptide also allows for establishing an assay in neuroblastoma cell lines rather than primary cells. Accordingly, the use of primary neurons as required in other cell based neurotoxin assays can be avoided. This is another advantage since the preparation of primary neurons is cumbersome and inefficient.

It is to be understood that the definitions and explanations of the terms made above apply mutatis mutandis for all aspects described in this specification in the following except as otherwise indicated.

The present invention also relates to a vector comprising the polynucleotide of the invention.

The term “vector”, preferably, encompasses phage, plasmid, viral or retroviral vectors as well as artificial chromosomes, such as bacterial or yeast artificial chromosomes. Moreover, the term also relates to targeting constructs which allow for random or site-directed integration of the targeting construct into genomic DNA. Such target constructs, in an aspect, comprise DNA of sufficient length for either homologous or heterologous recombination as described in detail below. The vector encompassing the polynucleotides of the present invention, in an aspect, further comprises selectable markers for propagation and/or selection in a host cell. The vector may be incorporated into a host cell by various techniques well known in the art. For example, a plasmid vector can be introduced in a precipitate such as a calcium phosphate precipitate or rubidium chloride precipitate, or in a complex with a charged lipid or in carbon-based clusters, such as fullerens. Alternatively, a plasmid vector may be introduced by heat shock or electroporation techniques. Should the vector be a virus, it may be packaged in vitro using an appropriate packaging cell line prior to application to host cells. Retroviral vectors may be replication competent or replication defective. In the latter case, viral propagation generally will occur only in complementing host/cells.

Moreover, in an aspect of the invention, the polynucleotide is operatively linked to expression control sequences allowing expression in prokaryotic or eukaryotic host cells or isolated fractions thereof in the said vector. Thus, in an aspect, the vector is an expression vector. Expression of the polynucleotide comprises transcription of the polynucleotide into a translatable mRNA. Regulatory elements ensuring expression in host cells are well known in the art. In an aspect, they comprise regulatory sequences ensuring initiation of transcription and/or poly-A signals ensuring termination of transcription and stabilization of the transcript. Additional regulatory elements may include transcriptional as well as translational enhancers. Possible regulatory elements permitting expression in prokaryotic host cells comprise, e.g., the lac-, trp- or tac-promoter in E. coli, and examples for regulatory elements permitting expression in eukaryotic host cells are the AOX1- or the GAL1-promoter in yeast or the CMV-, SV40-, RSV-promoter (Rous sarcoma virus), CMV-enhancer, SV40-enhancer or a globin intron in mammalian and other animal cells. Moreover, inducible expression control sequences may be used in an expression vector encompassed by the present invention. Such inducible vectors may comprise tet or lac operator sequences or sequences inducible by heat shock or other environmental factors. Suitable expression control sequences are well known in the art. Beside elements which are responsible for the initiation of transcription such regulatory elements may also comprise transcription termination signals, such as the SV40-poly-A site or the tk-poly-A site, downstream of the polynucleotide. In this context, suitable expression vectors are known in the art such as Okayama-Berg cDNA expression vector pcDV1 (Pharmacia), pBluescript (Stratagene), pCDM8, pRc/CMV, pcDNA1, pcDNA3 (Invitrogen) or pSPORT1 (Invitrogen). Preferably, said vector is an expression vector and a gene transfer or targeting vector. Expression vectors derived from viruses such as retroviruses, vaccinia virus, adeno-associated virus, herpes viruses, or bovine papilloma virus, may be used for delivery of the polynucleotide or vector of the invention into a targeted cell population. Methods which are well known to those skilled in the art can be used to construct recombinant viral vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994).

Moreover, the present invention relates to a host cell comprising the polynucleotide or the vector of the present invention.

The term “host cell” as used herein encompasses prokaryotic and eukaryotic host cells. In an aspect the host cell is a bacterial cell. In one aspect, the said bacterial host cell is an E. coli host cell. Such a bacterial host cell may be used, e.g., for reproduction of the polynucleotide or the vector of the present invention.

A eukaryotic host cell, in an aspect, is a cell which comprises the polypeptide and either the polynucleotide or the vector of the present invention wherein said polynucleotide or vector are expressed in the host cell in order to generate the polypeptide. The polynucleotide may be introduced into a host cell either transiently or stably. In an aspect, the eukaryotic host cell may be a cell of a eukaryotic host cell line which stably expresses the polynucleotide of the invention. In another aspect, the host cell is a eukaryotic host cell which has been transiently transfected with the polynucleotide or vector of the invention and which expresses the polynucleotide of the invention. In an aspect the host cell is a eukaryotic host cell which is capable of translocating a neurotoxin polypeptide into its cytoplasm. In an aspect, a cell capable of uptaking neurotoxin polypeptides can be a cell produces endogenously all necessary components for the neurotoxin polypeptide uptake. In an aspect, the said cell is a neuronal cell. In an aspect, the said cell is selected from the group consisting of: neuroblastoma cell lines, embryonic stem cells, and in an aspect non-human embryonic stem cells, induced pluripotent stem cells, and primary neurons, e.g. SH-SY5Y, SiMa, PC12, CHP134, LA-N-5, SK-N-BE(2), and the like. In another aspect, the said cell is a cell which has been genetically engineered to produce the components necessary for the neurotoxin polypeptide uptake. How such cells can be genetically engineered by molecular biology techniques is well known to the skilled person.

The present invention relates to a polypeptide encoded by the polynucleotide of the present invention.

The term “polypeptide” as used herein encompasses isolated or essentially purified polypeptides being essentially free of other host cell polypeptides. The term, in another aspect, includes polypeptide preparations comprising the polypeptide of the present invention and other proteins in addition. Moreover, the term includes, in an aspect, chemically modified polypeptides. Such modifications may be artificial modifications or naturally occurring modifications. The polypeptide of the present invention shall have the biological properties referred to above. The polypeptide of the invention, in an aspect, can be manufactured by chemical synthesis or recombinant molecular biology techniques well known for the skilled artisan. In an aspect, such a method of manufacturing the polypeptide of the invention comprises (a) culturing the host cell of the present invention described elsewhere herein in more detail and (b) obtaining from the said host cell the polypeptide of the present invention. In an aspect of this method, the polypeptide can be obtained by conventional purification techniques from a host cell lysate including affinity chromatography, ion exchange chromatography, size exclusion chromatography and/or preparative gel electrophoresis.

The present invention relates to a method for determining a proteolytically active neurotoxin polypeptide in a sample comprising:
a) contacting the host cell of or the polypeptide of the invention with a sample suspected to comprise said proteolytically active neurotoxin polypeptide under conditions which allow for proteolytic cleavage of the single chain luciferase fusion protein into separate LuxB and LuxA subunits;
b) allowing the said LuxB and LuxA subunit to form a biologically active luciferase; and
c) determining the said luciferase.

The method of the present invention can be assisted by automation. Specifically, in an aspect, step a) and/or b) may be assisted by robotic devices and automated reader systems for mixing compounds and measuring the luciferase activity. Suitable systems are known in the art and depend on the type of response to be determined. Moreover, the method may comprise additional steps pertaining to the sample preparation or generation of the polypeptide of the present invention.

The term “contacting” as used herein refers to bringing at least two different compounds in physical proximity as to allow physical and/or chemical interaction of said compounds. In the aforementioned method, the polypeptide according to the present invention is contacted with a sample suspected to comprise a biologically active neurotoxin polypeptide. The polypeptide shall be contacted for a time and under conditions sufficient to allow cleavage of the neurotoxin cleavage site in the polypeptide of the present invention by the neurotoxin polypeptide comprised by the sample. Contacting as used herein, in an aspect, occurs in a host cell of the present invention containing the polypeptide of the present invention. Thus, in an aspect, said polypeptide is comprised by a host cell and, in an aspect, the host cell of the present invention. The said time and conditions will dependent on the amount of neurotoxin polypeptide comprised by the sample as well as on the uptake of the neurotoxin polypeptide by the host cell. The person skilled in the art is well aware of which conditions need to be applied dependent on the host cell, kind of sample, and kind of neurotoxin which shall be determined. In another aspect, contacting occurs in a cell free system comprising the polypeptide of the invention as well as a substrate of the polypeptide of the present invention. The cell free system shall allow for measuring the activity of the polypeptide of the present invention, i.e. luciferase activity, upon contacting the system with a sample and, thus, allows for determining the neurotoxin protease activity in said sample.

In an aspect, said luciferase is determined by measuring the enzymatic conversion of a luciferase substrate. The latter one can be measured in an aspect by detecting the intensity of the light emitted during the conversion reaction. Suitable systems for measuring the light emission that occurs during the conversion reaction catalyzed by luciferases are well known in the art and commercially available. Moreover, suitable substrates which can be used for the luciferases are also well known and commercially available. In another aspect, however, the luciferase can be measured either by determining the amount of the formed LuxA ad/or B subunits or the formed holoenzyme. In an aspect, this determination is carried out by an antibody-based immunoassay using antibodies which specifically recognize a subunit or the holoenzyme, e.g., immunoblots or ELISA, or by SDS PAGE.

The term “sample” refers to a sample suspected to comprise neurotoxin polypeptide. The sample, in an aspect, is an aqueous solution. Such a sample may be a biological sample or may be a sample of an artificially generated aqueous solution. Such solutions, in an aspect, are obtained at different stages during neurotoxin manufacture, either for quality control and/or activity determination/specification purposes or for safety control. It is envisaged that the neurotoxin present in the said sample shall exhibit at least the neurotoxin protease activity. In another aspect, the neurotoxin is fully biologically active. In an aspect the said fully biologically active neurotoxin is required for entering the cell and for activating the read out based on the single chain luciferase fusion polypeptide of the present invention. Accordingly, such a fully biologically active neurotoxin is to be applied if a host cell is to be contacted with the sample to be analyzed by the method of the invention. In another aspect, the sample to be applied for the method of the invention comprises neurotoxin polypeptides or fragments thereof which merely exhibit neurotoxin protease activity. Such neurotoxin polypeptides or fragments are, in an aspect, muteins of neurotoxin polypeptides comprising or consisting essentially of a proteolytically active light chain. It is to be understood that samples comprising neurotoxin polypeptides or fragments thereof which merely exhibit neurotoxin protease activity shall be used if the sample is to be contacted to a cell free system as specified elsewhere herein in detail.

The neurotoxin polypeptide in a sample can be determined quantitatively or qualitatively. For a qualitative determination, in an aspect of the invention, the presence or absence of a neurotoxin polypeptide is determined. For a quantitative detection, in an aspect, the amount of the neurotoxin polypeptide is determined. In an aspect, the quantitative determination encompasses a determination of the absolute amount or a relative amount, i.e. an amount which is normalized such that the amount found in different samples can be compared with each other. In an aspect, this can be achieved by comparison of a measured luciferase activity for a test sample to a calibration curve which is to be established by subjecting calibration samples having predetermined amounts of the neurotoxin polypeptide to the method of the present invention.

It will be understood that in an aspect, the neurotoxin cleavage site comprised in the single chain luciferase fusion polypeptide is recognized by the proteolytically active neurotoxin polypeptide to be determined in the sample. In yet an aspect, said neurotoxin polypeptide is selected from the group consisting of: BoNT/A, BoNT/B, BoNT/C1, BoNT/D, BoNT/E, BoNT/F, BoNT/G or TeNT.

In another aspect of the method of the invention, the activity of more than one neurotoxin polypeptide shall be determined. To this end, a host cell can be applied which comprises a polynucleotide according to the present invention encoding a first single chain luciferase fusion polypeptide comprising: (i) a LuxB subunit of a first luciferase, (ii) a linker comprising a neurotoxin cleavage site for a first neurotoxin polypeptide, and (iii) a LuxA subunit of said first luciferase and another polynucleotide according to the present invention encoding a second single chain luciferase fusion polypeptide comprising: (i) a LuxB subunit of a second luciferase, (ii) a linker comprising a neurotoxin cleavage site for a second neurotoxin polypeptide, and (iii) a LuxA subunit of said second luciferase. It will be understood that the first and the second neurotoxin polypeptides are different and recognize and cleave different cleavage sites. Moreover, in an aspect it will be understood that the first luciferase holoenzyme comprising said first LuxB and LuxA subunits and the second luciferase holoenzyme comprising said second LuxB and LuxA subunits utilize generate different light emissions which can be distinguished from each other, e.g., emission maxima at different wavelengths. In an aspect, said first luciferase holoenzyme and/or said second luciferase holoenzyme can be bound to a fluorescent protein, e.g. green fluorescent protein (GFP), yellow fluorescent protein (YFP), red fluorescent protein (RFP), blue fluorescent protein, and the like. In an aspect, the first subunits are from Vibrio harveyi and the second subunits referred to before are from Vibrio fischeri.

Thus, the method of the present invention allows for an efficient determination of a biologically active neurotoxin polypeptide in a sample and can, therefore, be applied in high throughput screenings or quality control approaches. Thanks to the method of the present invention, neurotoxin polypeptide determination can be automated due to the use of cell culture cells rather than primary neurons. Moreover, the use of stably transfected host cell lines allows for a comparable quality of the readout system, i.e. the host cell to be applied in the method of the invention. Accordingly, the method of the present invention may serve as an alternative or may at least significantly reduce animal testing in the context of neurotoxin polypeptide development or quality control.

Further encompassed by the present invention is the use, in general, of the polynucleotide, the vector, the host cell or the polypeptide of the invention for determining a proteolytically active neurotoxin polypeptide in a sample in vitro.

Finally, the present invention contemplates a kit for determining a proteolytically active neurotoxin polypeptide in a sample comprising the polynucleotide, the vector, the host cell and/or the polypeptide of the present invention and, preferably, a detection agent for measuring the enzymatic conversion of a luciferase substrate and a luciferase substrate.

The term “kit” as used herein refers to a collection of means comprising the polypeptide, the polynucleotide, the vector and/or the host cell of the present invention which are provided in separate or common vials in a ready to use manner for carrying out the method of the present invention. In an aspect, the kit comprises additional means for carrying out the method of the present invention, in an aspect, calibration standard solutions comprising neurotoxin polypeptide and/or means for measuring the luciferase activity such as detection agents for luciferase or substrates converted by luciferase. Furthermore, in an aspect, the kit comprises instructions for carrying out the method of the present invention. These instructions can be provided as a manual or can be in the form of an computer-implementable algorithm on a data storage medium which upon implementation is capable of governing one or more steps of the method of the invention. In an aspect, the kit is to be used for carrying out the method of the invention specified above.

All references cited in this specification are herewith incorporated by reference with respect to their entire disclosure content and the disclosure content specifically mentioned in this specification.

### FIGURES

FIG. 1 shows a schematic drawing of the single chain luciferase fusion polypeptide of the in vention, (A) prevention of the self interaction of the subunits by a specific order of the said subunits; (B) prevention of the self interaction by ankyrin repeats; (C) prevention of the self interaction by globular proteins.

FIG. 2 shows the expression of MRZ_LuxAB0 and MRZ_LuxBA3 in different E. coli expression strains at 22° C., whereby soluble and insoluble fractions were separated. Load per lane: ⅙×OD₆₀₀ unit with 1×OD₆₀₀ unit defined as 1 ml of a culture with an OD₆₀₀ of 1.0. Selected marker sizes are depicted to the left. A shows the 12% SDS-PAGE analysis of the expression of MRZ_LuxAB0 and MRZ_LuxBA3 in the E. coli strains BL21 and Rosetta (DE3)/pRARE2. Gels were stained with Coomassie Blue. B shows the Western-blot analysis of a 12% gel; detection was performed with anti-Strep-tag antibody, secondary antibody anti mouse-AP, developed with NBT/BCIP.

FIG. 3 shows the expression of MRZ_LuxAB0 and MRZ_LuxBA3 in E. coli expression strain BL21 at 16° C., whereby soluble and insoluble fractions were separated. Load per lane: ⅙×OD₆₀₀ unit with 1×OD₆₀₀ unit defined as 1 ml of a culture with an OD₆₀₀ of 1.0. Selected marker sizes are depicted to the left. A shows the 12% SDS-PAGE analysis of the expression of MRZ_LuxAB0 and MRZ_LuxBA3 in the E. coli strain BL21. Gels were stained with Coomassie Blue. B shows the Western-blot analysis of a 12% gel; detection was performed with anti-Strep-tag antibody, secondary antibody anti mouse-AP, developed with NBT/BCIP.

FIG. 4 shows E. coli strain BL21 LuxAB0 fed-batch fermentation. One 5 l fermentation was run to generate 62 g (wcw) biomass.

FIG. 5 shows. OD₆₀₀ throughout fed-batch fermentation of A E. coli strain BL21 LuxAB0 and B E. coli strain BL21 LuxBA1.

FIG. 6 shows SDS-PAGE and Western blotting of samples from before induction and at time of harvest of A E. coli strain BL21 LuxAB0 and B E. coli strain BL21 LuxBA1.

FIG. 7 shows Strep-tag affinity batch-purification of BL21-MRZ-LuxAB0 and BL21-MRZ-LuxBA1. Load per lane is 1.25 μl undiluted sample of fractions Load, FT1, and FT2, and 2.5 μl undiluted sample of Pool E1, Pool E2, and Pool E3. Selected marker sizes are depicted to the left. A SDS-PAGE analysis; Gel 12%, stained with Coomassie Blue. B Western-blot analysis; Gel 12%, detection with anti-Strep antibody (StrepMAB-Classic, HRP conjugate, IBA, Cat. No. 2-1509-001, dilution 1:10000 in PBS-Tween containing 2% BSA).

FIG. 8 shows the analysis of expression in small scale E. coli shake flask cultures. Target vectors were expressed in the host strains BL21(DE3) and BL21(DE3) Rosetta, and cultured at 37° C. Samples for analysis were drawn just before induction as well as 2, 4, and 20 hours after induction of target expression. Load per lane: 0.25×OD₆₀₀ units with 1×OD₆₀₀ unit defined as 1 ml of a culture with an OD₆₀₀ of 1.0. Left side shows BL21(DE3) and right side shows BL21(DE3) Rosetta. A shows LuxBA3-Strep (84.9 kDa), B GST-LuxBA3-Strep (111.2 kDa), and C 9×His-LuxBA3-Strep (86.8 kDa).

FIG. 9 shows the analysis of 9×His-Xa-LuxBA3-Strep protein expression. 1 l shake flask culture grown at 25° C. (before and after induction). 12% SDS-PAGE followed by Coll. Coomassie-staining and anti-Strep-tag Western blotting. Load per lane: 0.25×OD₆₀₀ units with 1×OD₆₀₀ unit defined as 1 ml of a culture with an OD₆₀₀ of 1.0. Marker: Fermentas PageRuler™ Prestained Protein Ladder.

FIG. 10 shows the analysis of final LuxBA3 protein samples. 12% SDS-PAGE and Coll. Coomassie staining. The two different gels refer to two independent FPLC runs (left panel: 1.89 mg total; right panel: 1.90 mg total) but from the same starting material.

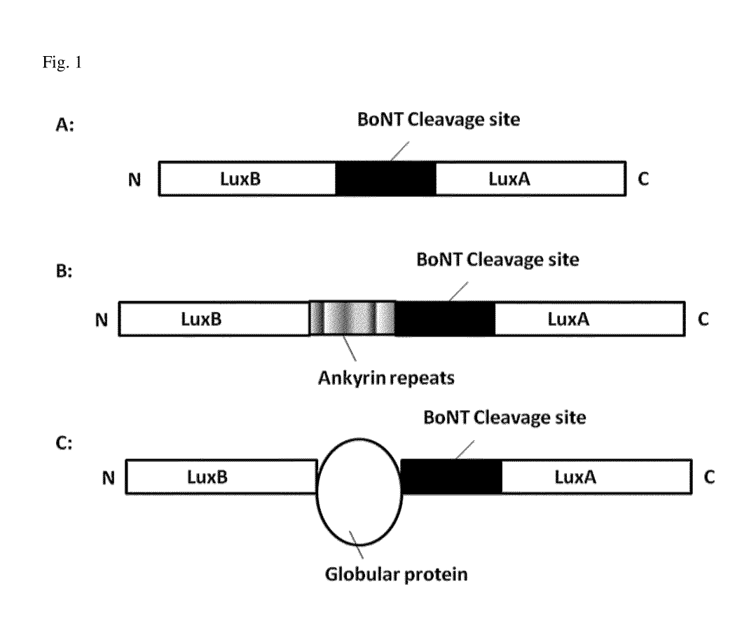

### EXAMPLES

The invention will now be illustrated by Examples which shall, however, not be construed as limiting the scope of the invention.

### Example 1

### Expression, Fermentation and Purification of Different Luciferase Constructs in Escherichia coli

Expression of MRZ_LuxAB0 and MRZ_LuxBA3 in E. coli

The constructs MRZ_LuxAB0 (SEQ ID NO: 9) and MRZ_LuxBA3 (SEQ ID NO: 10) are pASK-IBA3-plus vector constructs, encoding for two different Luciferase-targets both carrying a C-terminal Strep-tag II. The expected molecular weight of Luciferase AB0 carrying a C-terminal Strep-tag-II is 78.6 KDa, with an estimated pI of 5.22. The expected molecular weight of Luciferase BA3 carrying a C-terminal Strep-tag-II is 84.9 KDa, with an estimated pI of 5.20.

Expression of MRZ_LuxAB0 and MRZ_LuxBA3 at 22° C.

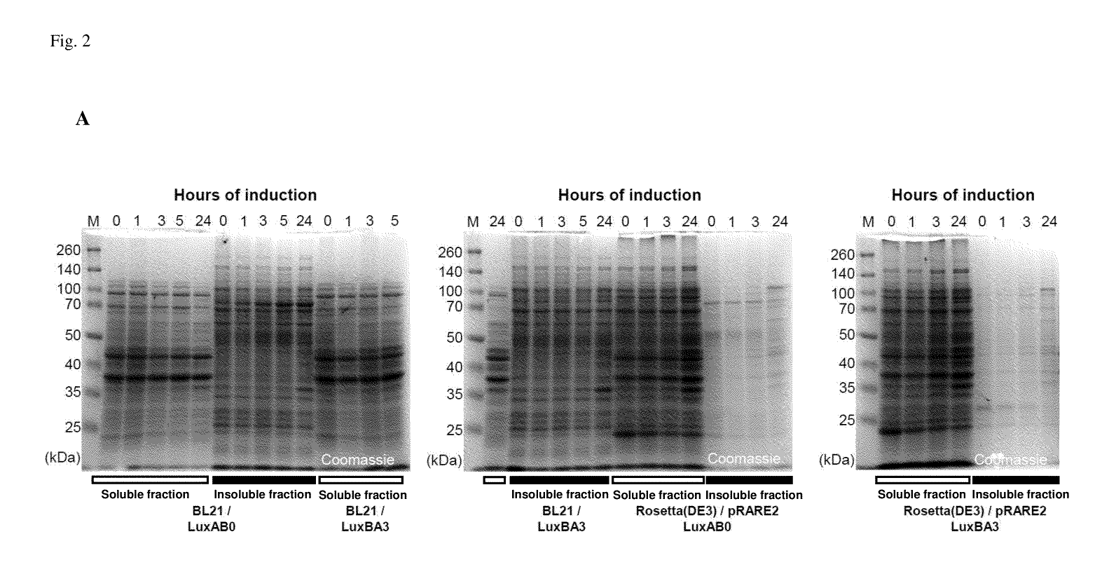

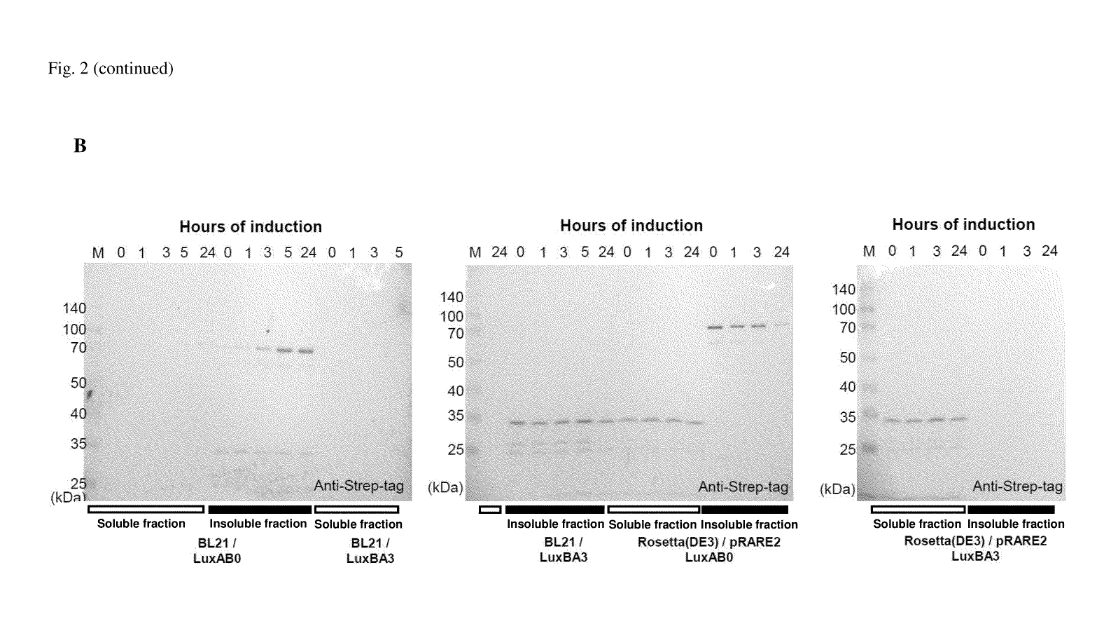

For analyzing the expression MRZ_LuxAB0 and MRZ_LuxBA3 (both Amp^{R}) were transformed into the E. coli strains BL21 and Rosetta (DE3)/pRARE2 (Cam^{R}). Cells were grown at 37° C. in LB medium supplemented with 200 g/ml ampicillin (and 30 μg/ml chloramphenicol for the respective strains) until an OD₆₀₀ of 0.4 (BL21) or an OD₆₀₀ of 0.2 (Rosetta (DE3)/pRARE2) was reached. Each culture was shifted to 22° C. and grown until an OD₆₀₀ of 0.65 (BL21) or an OD₆₀₀ of 0.3 (Rosetta (DE3)/pRARE2) was reached. Cultures were induced with 0.2 μg/ml anhydrotetracycline and grown for another 24 hours at 22° C. After 0, 1, 3, 5, and 24 hours of induction samples were taken and treated with Bug buster HT solution (Novagen) to break the cells and separate soluble and insoluble protein fractions. Samples were analyzed via SDS-PAGE analysis on 12% Gels (and via Western-blot with anti-Strep-tag antibody), as seen in FIGS. 2A and 2B.

Expression of MRZ_LuxAB0 and MRZ_LuxBA3 at 16° C.

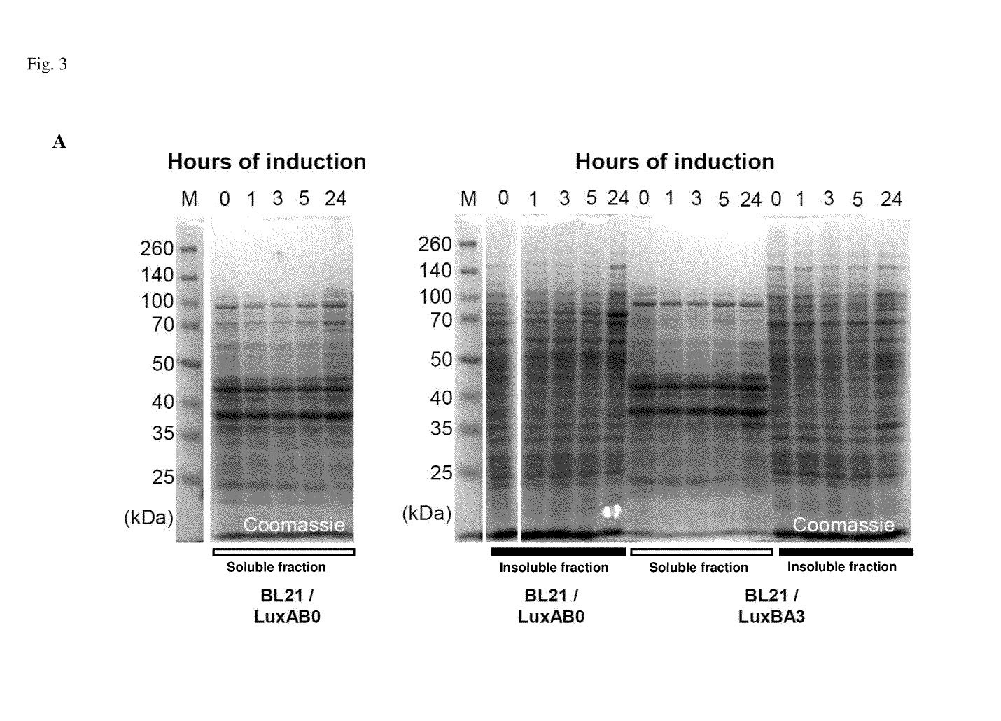

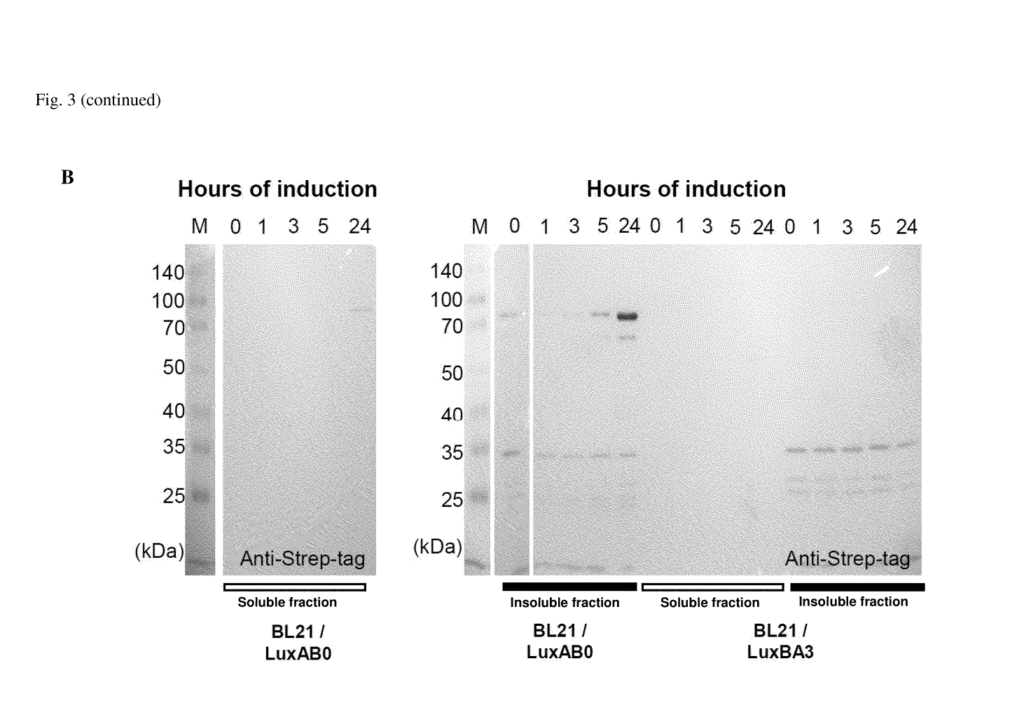

For analyzing the expression MRZ_LuxAB0 and MRZ_LuxBA3 (both Amp^{R}) were transformed into E. coli BL21. Cells were grown at 25° C. in LB medium supplemented with 200 μg/ml ampicillin until an OD₆₀₀ of 0.15 was reached. Each culture was shifted to 16° C. and grown until an OD₆₀₀ of 0.2 (MRZ_LuxAB0) or an OD₆₀₀ of 0.35 (MRZ_LuxBA3) was reached. Cultures were induced with 0.2 μg/ml anhydrotetracycline and grown for another 24 hours at 16° C. After 0, 1, 3, 5 and 24 hours of induction samples were taken and treated with Bug buster HT solution (Novagen) to break the cells and separate soluble and insoluble protein fractions. Samples were analysed via SDS-PAGE analysis on 12% Gels (and via Western-blot with anti-Strep-tag antibody), as seen in FIGS. 3A and 3B.

To clearly relate bands in the Coomassie stained gels shown in FIGS. 2A and 3A to the Luciferase AB0 and BA3 target and to estimate the amount of soluble compared to insoluble target, western blot analysis with anti-Strep-Tag antibody was performed. The western-blot analysis revealed the presence of a protein running between the 75 and the 100 kDa molecular weight marker band (corresponding with the expected molecular weight of 78.6 kDa of the translated Luciferase AB0 protein) in the insoluble protein fraction of the BL21/LuxAB0 expression, as shown in FIGS. 2B and 3B. The LuxAB0-construct showed the best soluble expression in BL21 after 24 hours of induction at 16° C. In both Figures no specific western-blot signal was detected by the anti-Strep-tag antibody in the LuxBA3 expression strains. In FIG. 3B, however, a very weak signal can be seen after 24 hours of expression in BL21 in the soluble protein fraction.

High Cell Density Batch-Fermentation of the E. coli Strains BL21 LuxAB0 and BL21 LuxBA1

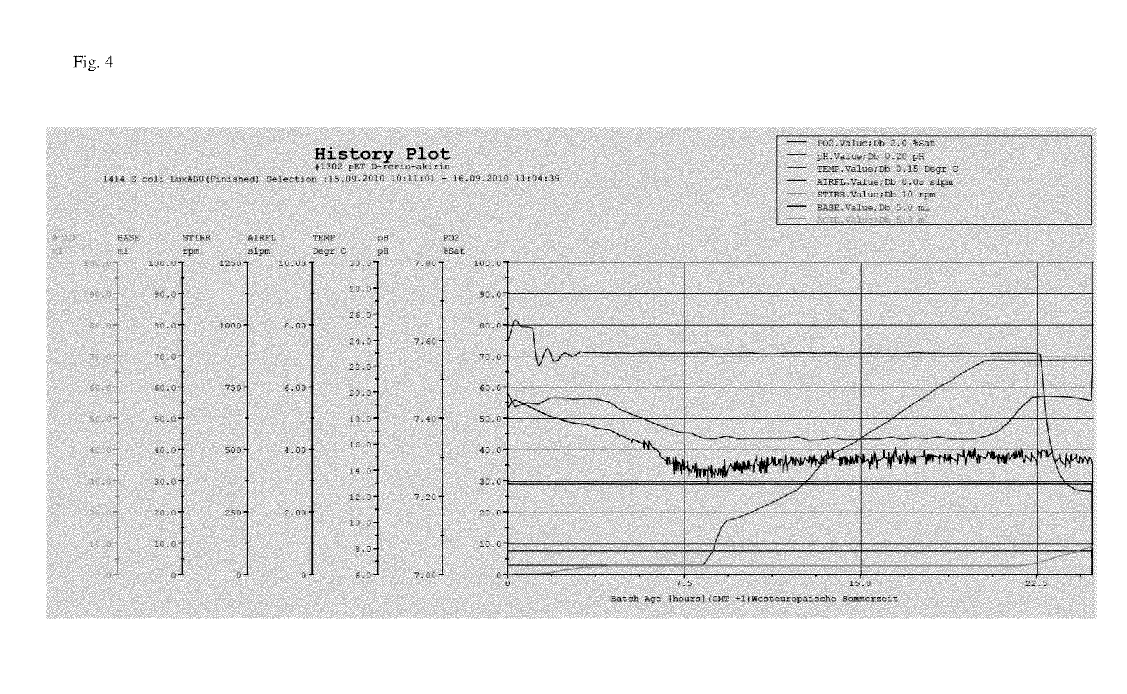

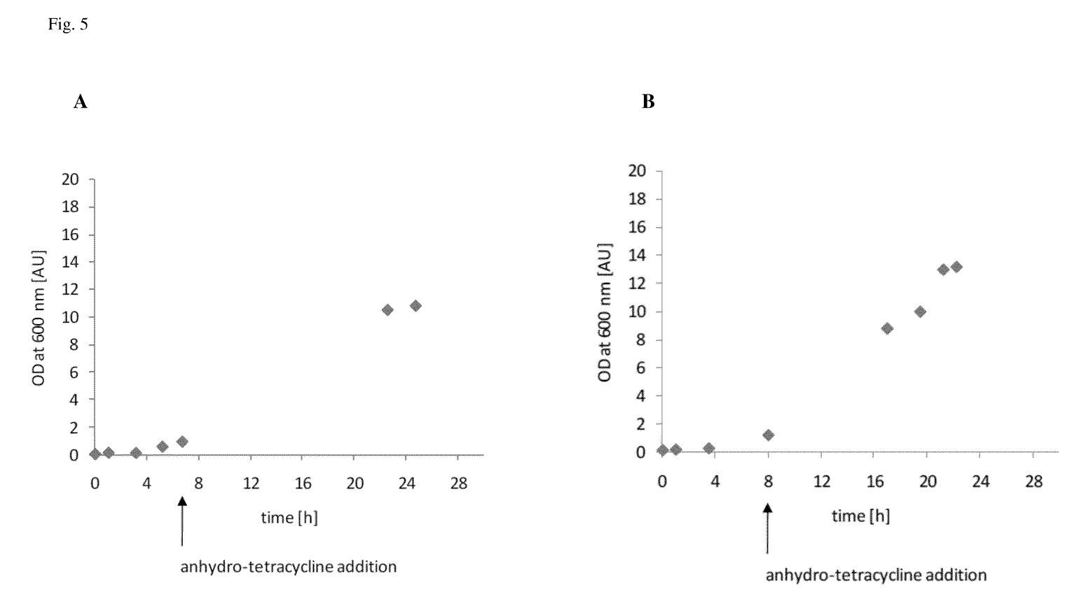

E. coli strains BL21 LuxAB0 and BL21 LuxBA1 were separately grown in batch fermentation mode. A single colony of each strain was picked from an LB plate, inoculated in 2×100 ml LB medium, grown at 25° C., 175 rpm for 16 hours. These preculture were used to inoculate 4.5 l fermentation medium. Ampicillin was added in all cultures to 100 μg/ml. Growth was recorded throughout the fermentation (FIGS. 4, 5A, and 5B). Fermenter settings are summarized in Table 1.

Table 1 summarizes fermenter settings.

Fermenter settings summary:

| | |
|---|---|
| Preculture volume | 125 ml |
| Initial fermentation volume | 4.5 l |
| pH | 7.4 (adjusted using ammonia/phosphoric |
| | acid) |
| pO₂ | 20% (stir → airflow cascade) |
| Temperatur | 23° C. whole cultivation period |
| Antifoam reagent | Antifoam A (Sigma) at 1 ml/l |
| Antibiotic | Ampicillin at 100 μg/ml |
| Inducer | Anhydro-tetracycline at 1 mg/l |

The fermentation medium was made as follows (per liter): For YTG base, to 900 ml of H₂O add 12 g bacto-tryptone, 24 g bacto-yeast extract, and 4 mL glycerol. In a separate flask dissolve in 90 mL H₂O 2.31 g KH₂PO₄ monobasic, 12.54 g K₂HPO₄ dibasic, and adjust volume to 100 mL with H₂O. Both solutions were autoclaved separately and mixed only after cooling down to below 60° C.

The fermenter cultures were inoculated to a starting OD₆₀₀ of about 0.1 at 23° C. which was kept throughout the whole fermentation process. The culture of E. coli strain BL21 LuxAB0 had reached an OD₆₀₀ of 0.96 after 7 hours, wherein the culture of E. coli strain BL21 LuxBA1 had reached an OD₆₀₀ of 1.2 after 8 hours. Then the inducer anhydro-tetracycline was added (1.0 mg/l final concentration). The cultures were harvested after an additional 18 hours (LuxAB0) or 15 hours (LuxBA1) by centrifugation at 8.000 g for 20 min at 4° C. The supernatant was discarded, the cell pellets snap frozen in liquid nitrogen and then stored at −80° C. until further use. The final OD₆₀₀ were 10.8 (LuxAB0) and 13.5 (LuxBA1) with a culture volume of about 5 l. The biomass yield were 62 g (wcw, LuxAB0) and 70 g (wcw, LuxBA1).

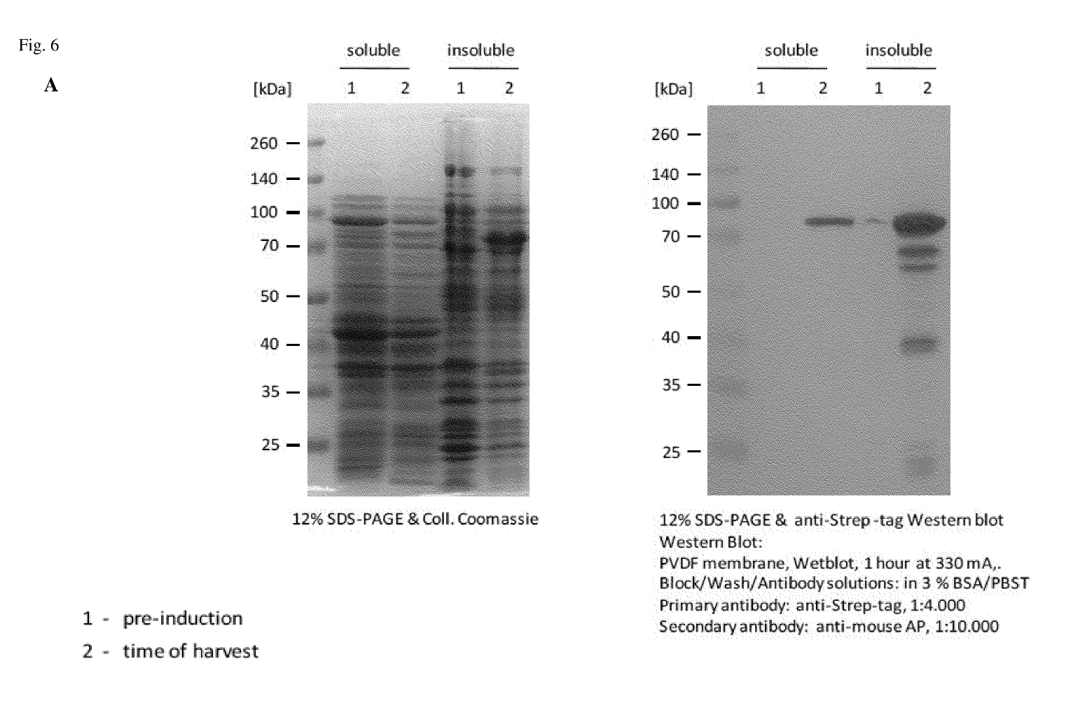

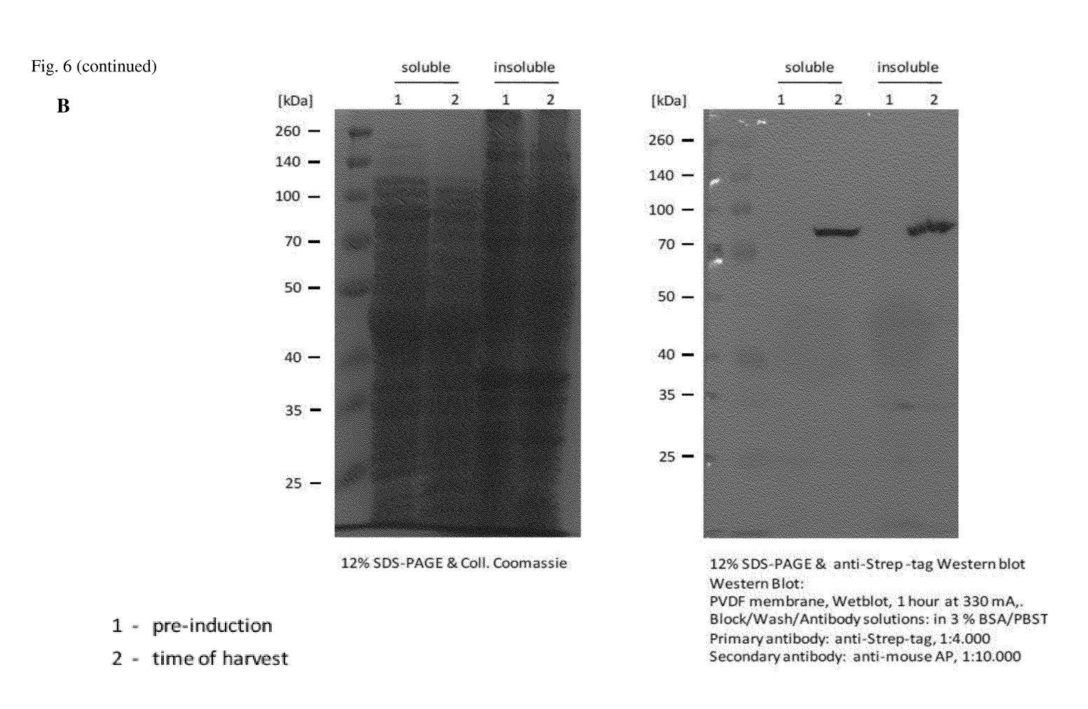

During the fermentation, two samples were drawn (just before anhydro-tetracycline addition and at the time of harvest), the cells pelleted by centrifugation, and then also stored at −80° C. until further use. These two samples were processed for analysis using Bugbuster (Novagen) to separate soluble from insoluble material. Comparable amounts were analyzed by SDS-PAGE and subsequent Colloidal Coomassie staining and Western blotting, respectively (FIGS. 6A and 6B; load per lane: 0.25×OD₆₀₀ units for Coomassie staining and 0.5×OD₆₀₀ units for anti-Strep-tag Western blotting, with 1×OD₆₀₀ unit defined as 1 ml of a culture with an OD₆₀₀ of 1.0.)

Strep-Tag Affinity Batch-Purification of LuxAB0 and LuxBA1

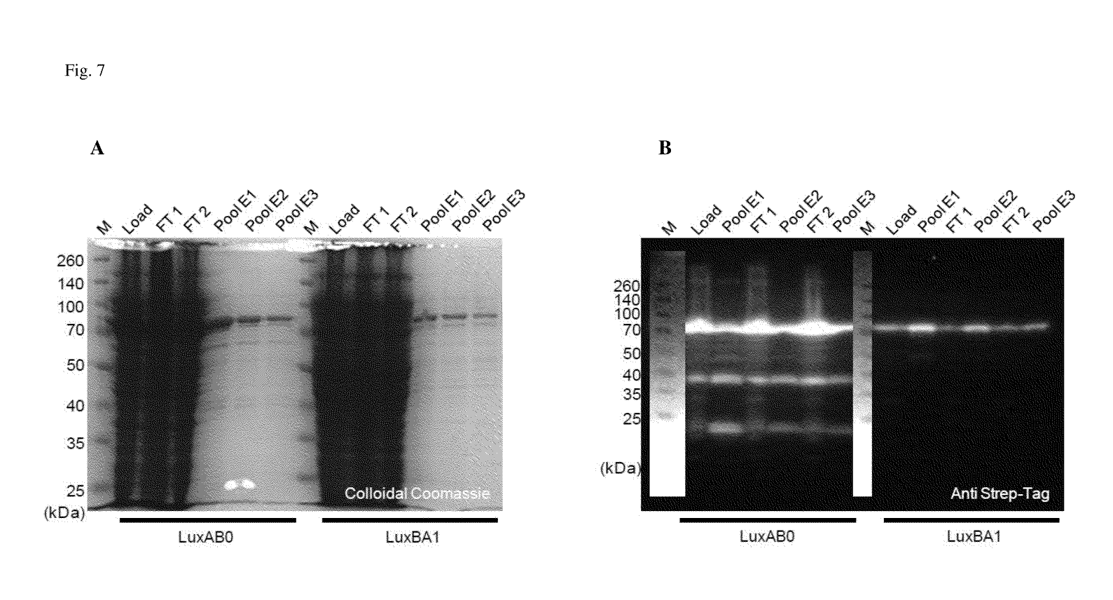

62 g (wcw, LuxAB0) fermenter biomass or 70 g (wcw, LuxBA1) fermenter biomass were resuspended in 150 ml resuspension buffer (100 mM Tris/HCl pH 8.0, 150 mM NaCl, and 1 mM EDTA). The cells were broken by passing them two times through a microfluidizer. Unbroken cells were removed by centrifugation at 4° C., 10000×g for 30 minutes (pellet was discarded, supernatant=Load). 1 ml (bed volume) Strep-Tactin Superflow matrix from IBA was added to the crude extract (supernatant, Load) and binding was performed for 30 minutes with gentle shaking at 4° C. The suspension was centrifuged at 4° C., 2000×g for 10 minutes. The matrix was transferred to a gravity column, the flowthrough was collected (FT1). The column was washed one time with 5 ml of resuspension buffer (wash was added to the FT1) followed by six elution steps with 500 μl resuspension buffer containing 2.5 mM D-desthiobiotin (E1-E6, first Elution). The flow-trough (FT1) was loaded on the column again. The flow-through of this step was collected again (FT2). The column was washed one time with 5 ml of resuspension buffer (wash was added to the FT2). A second elution was performed consisting of six elution steps with 500 μl resuspension buffer containing 2.5 mM D-desthiobiotin (E1-E6, second Elution). The second flow-trough (FT2) was loaded on the column again. A third elution was performed consisting of six elution steps with 500 μl resuspension buffer containing 2.5 mM D-desthiobiotin (E1-E6, third Elution). The following elution fractions were pooled: Pool E1 (E1-E6 from elution 1), Pool E2 (E1-E6 from elution 2), and Pool E3 (E1-E6 from elution 3). Samples were analysed via 12% SDS-PAGE analysis and western blot using Strep-tag antibody (StrepMAB-Classic, HRP conjugate, IBA, Cat. No. 2-1509-001), as shown in FIG. 7.

Protein concentrations of the elution fractions were determined using Bradford analysis. Each elution fraction pool was split in two halves (with 1.5 ml each respectively) and stored at 4° C. The total protein yield of the elution fractions were approximately 6 mg for the LuxAB0 construct (purified out of 62 g wcw fermenter biomass) and approximately 3 mg for the LuxBA1 construct (purified out of 70 g wcw fermenter biomass), as shown in Table 2.

Table 2 shows total protein yield for LuxAB0 and LuxBA1 constucts.

| | Concentration | | |
|---|---|---|---|
| Eluation pool | [μg/μl] | Volume [ml] | Total protein yield [mg] |
| LuxAB0 E1 | 1.00 | 3.00 | 3.00 |
| LuxAB0 E2 | 0.53 | 3.00 | 1.59 |
| LuxAB0 E3 | 0.45 | 3.00 | 1.35 |
| LuxBA1 E1 | 0.40 | 3.00 | 1.20 |
| LuxBA1 E2 | 0.36 | 3.00 | 1.08 |
| LuxBA1 E3 | 0.25 | 3.00 | 0.75 |

### Example 2

### Cloning of Different LuxBA3-Strep-Tag Expression Vectors and Expression in E. coli

Cloning of Expression Vectors

Using the template DNA (SEQ ID NO: 10), the target sequence was amplified and subcloned into pET-based expression vectors. The resulting target vectors were named accordingly (Table 3). E. coli transformants were screened, and plasmid DNA from several candidates was isolated and sequenced. Their target sequences were verified by DNA sequencing.

Table 3 shows nomenclature and SEQ ID NOs. of generated expression vectors. Abreviations: His, Histidin; Strep, Streptavidin-tag; Kan, Kanamycin; Amp, Ampicillin.

| | Protein features (N- to | | | DNA vector |
|---|---|---|---|---|
| Expression vector | C-terminal) | Resistance | Protein sequence | sequence |
| pTZ_E02_LuxBA3 | LuxBA3-Strep | Kan | SEQ ID NO: 11 | |
| pTZ_E30_LuxBA3 | GST-LuxBA3-Strep | Amp | SEQ ID NO: 12 | |
| pTZ_E47_LuxBA3 | 9xHis-LuxBA3-Strep | Kan | SEQ ID NO: 13 | SEQ ID NO: 14 |

Expression in Small Scale E. coli Shake Flask Cultures

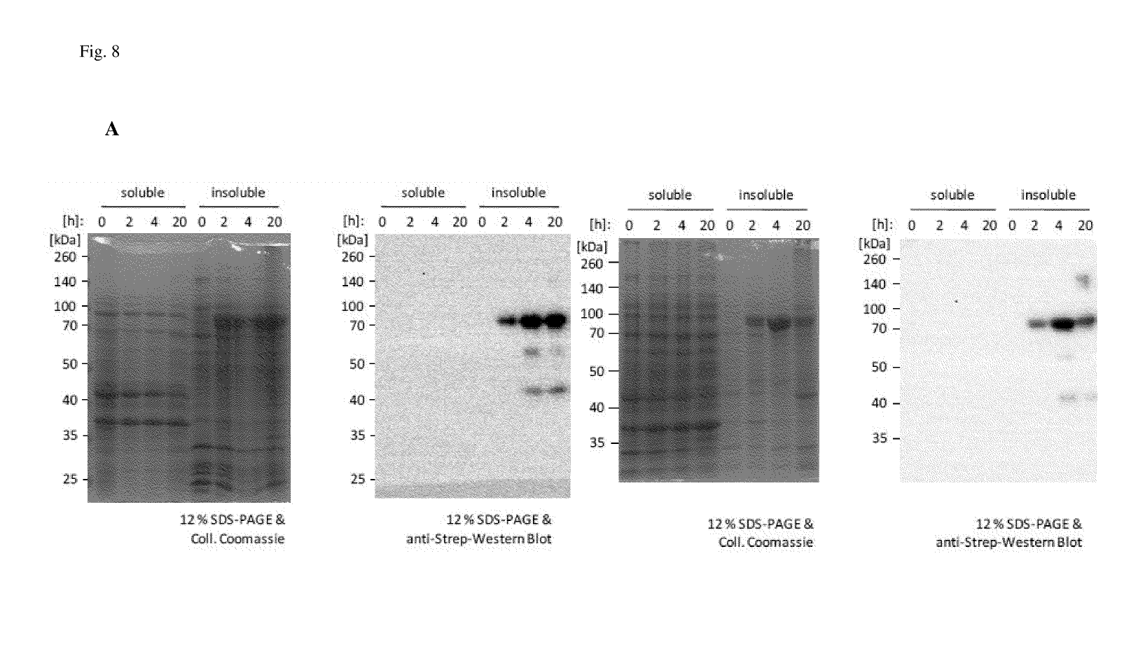

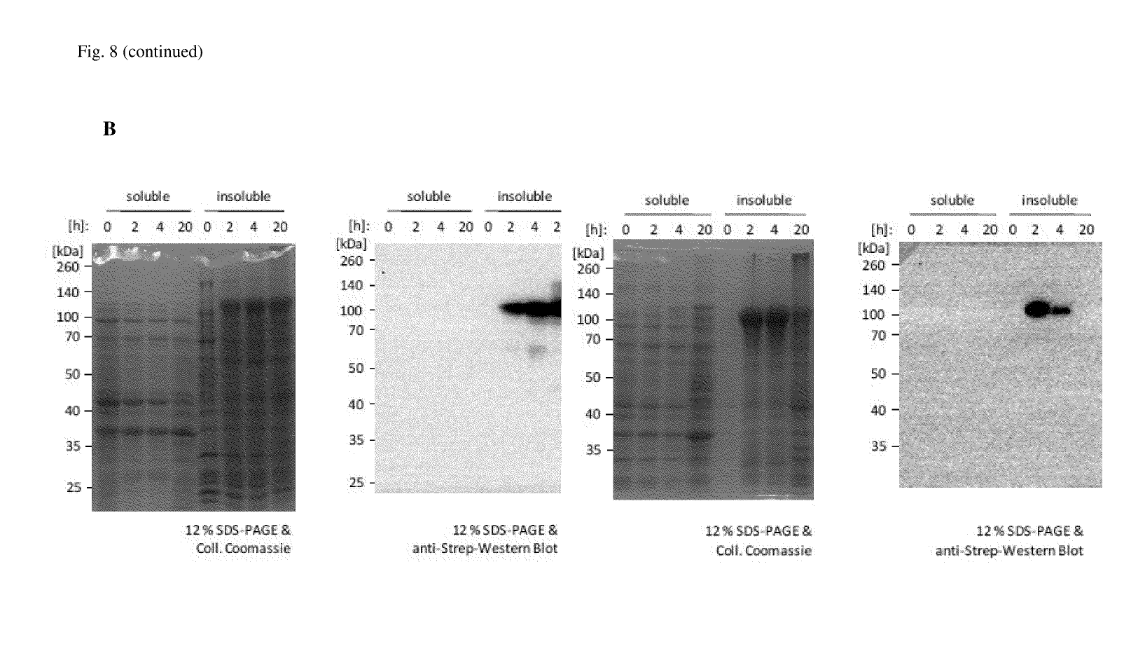

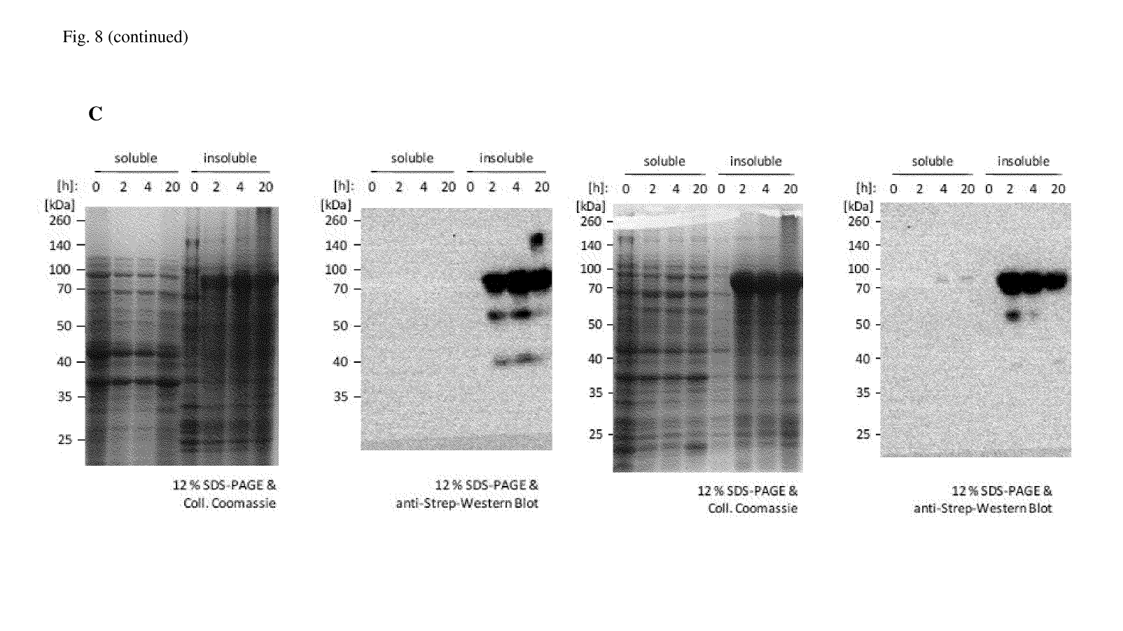

Each of the three constructs listed in Table 3 were expressed in two different host strains. Samples for analysis were drawn at 4 timepoints (just before IPTG addition as well as 2, 4, and 20 hours post-induction). A single colony was picked from an LB plate, inoculated in 5 ml LB medium (incl. the appropriate antibiotics), and grown overnight at 37° C., 175 rpm. From these, fresh 30 ml LB cultures were inoculated to a starting OD₆₀₀ of 0.1. When the cultures reached an OD₆₀₀ of about 0.4, each culture was kept at 37° C. and target expression was induced in all cultures by the addition of IPTG (0.5 mM). All samples were processed in the same manner using Bugbuster (Novagen) to separate soluble from insoluble material. Comparable amounts were analyzed by SDS-PAGE and subsequent Colloidal Coomassie staining and anti-Strep Western blotting, respectively (FIGS. 8A-C).

### Example 3

### Expression of LuxBA3 Construct and Purification of LuxBA3 Protein

Expression of the LuxBA3 Construct

The construct pTZ_E47_LuxBA3 (9×His-Xa-LuxBA3-Strep protein; see Table 3) was expressed in E. coli strain BL21(DE3). A single colony was picked from an LB plate, inoculated in 5 ml LB medium (incl. Kanamycin, 25 μg/ml), and grown overnight at 25° C., 175 rpm. On the next morning, 100 ml LB shake flask cultures were inoculated to a starting OD₆₀₀ of 0.1. When the cultures reached an OD₆₀₀ of about 0.6, target expression was induced in all cultures by the addition of IPTG (0.02, 0.10, and 0.25 mM IPTG, respectively). Samples for analysis were drawn just before IPTG addition and 20 hours post-induction. These expression conditions were used to generate additional biomass (3×11).

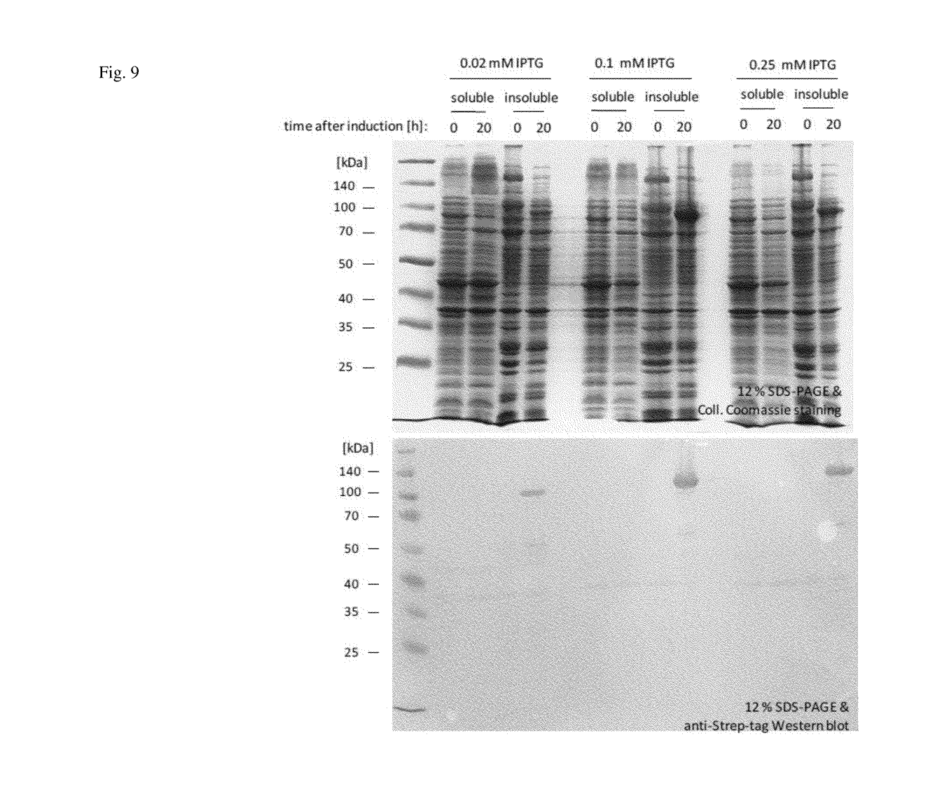

All samples were processed in the same manner using Bugbuster (Novagen) to separate soluble from insoluble material. Comparable amounts were analyzed by SDS-PAGE and subsequent Colloidal Coomassie staining and Western blotting, respectively (FIG. 9). Beyond the sampling over the time course, the cultures were harvested 20 hours post-induction by centrifugation at 5.000 g for 15 min. Cell pellets were stored at −20° C.

Purification of the Target Protein 9×His-Xa-LuxBA3-Strep (87 kDa) from the Insoluble Fraction Under Denaturing Conditions—Refolding on Column

For the purification from the insoluble fraction, all three cell pellets (see above) were combined and processed at once. After binding of the denatured target protein on a NiNTA chromatography column, the target was refolded on column and then eluted. Final yield of purification was 3.8 mg with an estimated purity of 90%.

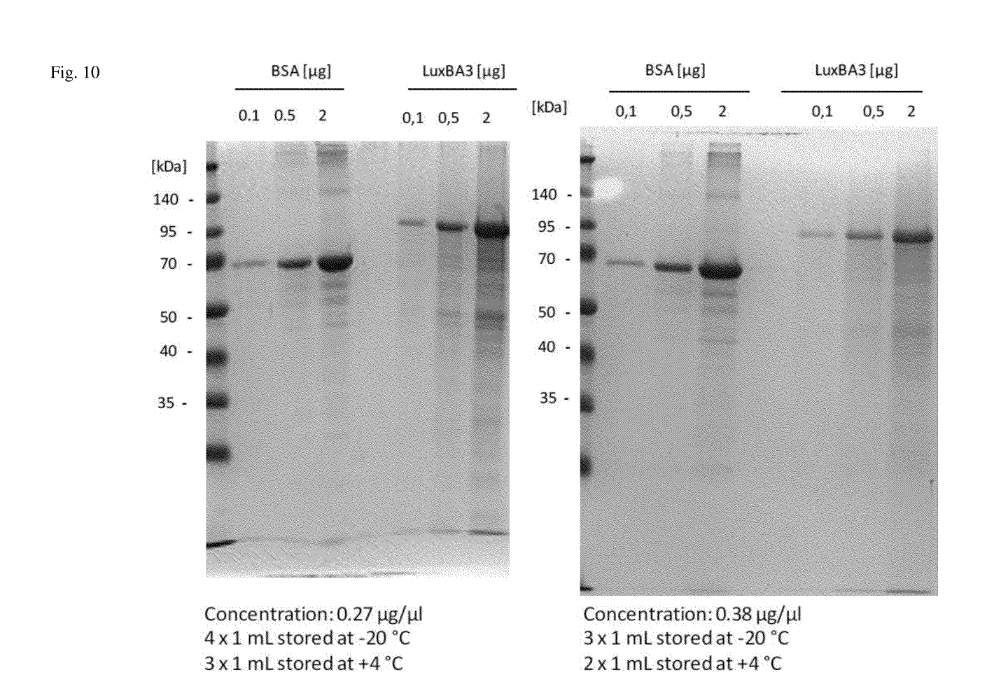

The following protocol was used to solubilize the insoluble protein fraction: The biomass was resuspended in PBS pH 7.4 including protease inhibitors. Mechanical cell lysis was performed by passing the resuspended biomass through a microfluidizer. Cell were centrifuged to separate insoluble and soluble fraction. After centrifugation in PBS, the pellet (insoluble fraction) was resuspended, urea was added to 8 M final concentration, and the mixture was incubated for one hour at room temperature with stirring. After a centrifugation at room temperature an urea-insoluble (pellet) and an urea-soluble fraction (supernatant) were obtained. The urea-soluble fraction (supernatant) was loaded onto Nickel-chelating resin (FPLC) using a loading buffer containing 8 M urea). In the next step a linear gradient starting from 8 M to 0 M urea follows over 2 hours. After washing with PBS (no urea from this step onwards) a second washing with PBS and 20 mM Imidazole followed. The proteins are elutated by a linear gradient from 20 to 500 mM Imidazole (in PBS). The final samples were analyzed by SDS-PAGE and Coll. Coomassie staining (FIG. 10). Purified target protein was stored in PBS pH 7.4, residual imidazole. Samples were aliquoted. About one half each was stored at +4° C. and the other half frozen and stored at −20° C.

Further, analysis of the 9×His-Xa-LuxBA3-Strep protein revealed that the protein is cleavable by BoNT/A activity but that this cleavage did not result in any luciferase activity as desired.

1-15. (canceled)

16. A polynucleotide encoding a single chain luciferase fusion polypeptide comprising: (i) a LuxB subunit, (ii) a linker comprising a neurotoxin cleavage site, and (iii) a LuxA subunit.

17. The polynucleotide of claim 16, wherein the luciferase LuxA subunit and/or LuxB subunit are from Vibrio fischeri or Vibrio harveyi.

18. The polynucleotide of claim 16, wherein the neurotoxin cleavage site is selected from the group consisting of a neurotoxin cleavage site from SNAP-25, a neurotoxin cleavage site from VAMP, a neurotoxin cleavage site from syntaxin, and the autocatalytic cleavage site from the neurotoxin polypeptide.

19. A vector comprising the polynucleotide of claim 16.

20. The vector of claim 19, wherein the vector is an expression vector.

21. A host cell comprising the vector of claim 19.

22. A host cell comprising the polynucleotide of claim 16.

23. The host cell of claim 22, wherein the host cell is a cell capable of translocating a neurotoxin polypeptide into its cytoplasm.

24. The host cell of claim 23, wherein the host cell is selected from the group consisting of neuroblastoma cell lines and primary neurons.

25. A polypeptide encoded by the polynucleotide of claim 16.

26. A method for determining proteolytic activity of a neurotoxin polypeptide in a sample comprising:
a) contacting the host cell of claim 22 with a sample suspected to comprise proteolytically active neurotoxin polypeptide under conditions which allow for proteolytic cleavage of a single chain luciferase fusion protein into separate LuxB and LuxA subunits;
b) allowing the LuxB and LuxA subunits to form a biologically active luciferase; and
c) determining luciferase activity.

27. A method for determining proteolytic activity of a neurotoxin polypeptide in a sample comprising:
a) contacting the polypeptide of claim 25 with a sample suspected to comprise the proteolytically active neurotoxin polypeptide under conditions which allow for proteolytic cleavage of a single chain luciferase fusion protein into separate LuxB and LuxA subunits;
b) allowing the LuxB and LuxA subunits to form a biologically active luciferase; and
c) determining luciferase activity.

28. The method of claim 26, wherein the luciferase activity is determined by measuring enzymatic conversion of a luciferase substrate.

29. The method of claim 26, wherein a neurotoxin cleavage site of the single chain luciferase fusion polypeptide is recognized by a proteolytically active neurotoxin polypeptide in the sample.

30. The method of claim 26, wherein the neurotoxin polypeptide is selected from the group consisting of Clostridium botulinum toxin type A (BoNT/A), Clostridium botulinum toxin type B (BoNT/B), Clostridium botulinum toxin type C1 (BoNT/C1), Clostridium botulinum toxin type D (BoNT/D), Clostridium botulinum toxin type E (BoNT/E), Clostridium botulinum toxin type F (BoNT/F), Clostridium botulinum toxin type G (BoNT/G) and Clostridium tetani tetanus toxin (TeNT).

32. A kit for determining proteolytic activity of a neurotoxin polypeptide in a sample comprising, the host cell of claim 22, a luciferase substrate, and a detection agent for measuring enzymatic conversion of the luciferase substrate.

33. A kit for determining proteolytic activity of a neurotoxin polypeptide in a sample comprising, the polypeptide of claim 25, a luciferase substrate, and a detection agent for measuring enzymatic conversion of the luciferase substrate.
